(19) United States
(12) Patent Application Publication
(10) Pub. No.: US 2010/0068747 A1
(43) Pub. Date: Mar. 18, 2010

(54) Generic Assay for Monitoring Endocytosis

(75) Inventor: Kurt HERRENKNECHT, Hamburg (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Evotec Technologies GmbH

(21) Appl. No.: 12/095,774

(22) PCT Filed: Dec. 1, 2006
(86) PCT No.: PCT/EP06/69216

§ 371 Date: Dec. 29, 2008

(30) Foreign Application Priority Data
Dec. 1, 2005 (EP) 05111595.4

(51) Int. Cl. C12Q 1/02

(52) U.S. Cl. 435/29

(57) ABSTRACT

A method for monitoring the internalisation of a cell surface molecule of interest is provided utilizing a detectable lectin.

In drug development there is a constant trend to obtain more information as early as possible in the drug discovery process and this constantly demands for novel approaches extending by far simple binding assays and enzymological assays as being used in the past. Part of the reason for this is the frequent failure of drug candidates in the late stages of clinical trials and the high costs and loss in time associated with this. This development ultimately gave rise to the field of high-content screening (HCS), which is expected to yield a surplus of information on a compound and its cellular mode of action over simple Boolean binding data as this information will be obtained at very early stages of compound screening and thus might be used to rule out a compound which might turn out to be problematic at later stages of the clinical development.

The term high-content screening usually refers to an (automated) multi-parameter analysis to capture a set of read-out variables from a (live) cell-based assay in a microtiter format (Dove, 2003). In this regard, functional assays in live cells represent a class of analytical techniques that have been developed and miniaturized to meet this demand. They ideally allow for the correlation of key read-out parameters—like functional property, target affinity, and toxicity—with the characteristics of compound molecules in order to define promising lead structures and discard compounds with less suitable features very early in the screening process. Systematic advantages over the conventional high-throughput binding screening approach can be attributed to the presentation of the target in a cellular context, which models serum binding and, thus, includes membrane barriers and cellular metabolism. However, time consuming cell culturing, population heterogeneity, and cell sensitivity to other treatment effects are major drawbacks of the approach (Shoemaker et al., 2002). The most relevant applications for HCS are signaling pathway analysis, multi-parameter (multiplexed) assays, morphological change analysis, translocation and G protein-coupled receptor (GPCR) assays (Comley, 2005). In this context, spectrometric techniques have proven most versatile to assess signal response and the majority of all assays integrate a fluorescence-based detection to measure the signal of choice. Furthermore, fluorescence microscopy provides a convenient read-out method since imaging and image processing can be multiplexed and automated—which makes it compatible with high-throughput—and additionally assesses spatial information, adding on a level of information (Mitchison, 2004).

Being generally used as secondary screens for the validation of compounds identified in primary HTS, cell-based assays are being predicted to move to the front position in the screening process and to gain a 50% increase in the number of screens run in the biotech and pharmaceutical industry over the next years, according to a recent survey (Comley, 2005). In order to meet the demand for highly sensitive fluorescence detection in a high throughput mode devices have been developed that are able to automatically capture fluorescence images from cell cultures in microtiter-plates and process the images on-line to extract the signal of choice. One such device is the Opera™ from Evotec Technologies GmbH which is a microplate imager reader that allows for simultaneous multiple laser-based excitation confocal imaging and on-the-fly image processing and analysis by use of Evotec Technologies' proprietary Acapella™ software.

GPCRs are a broad class of receptors, which is represented by a superfamily of 800 to 1,000 genes in the human genome (Eglen, 2005). All of them contain seven membrane-spanning regions with their N-terminus on the exoplasmic face and the C-terminus on the cytosolic face. Ligand binding induces a conformational change in the structure and permits binding of a trimeric G protein, which in turn promotes exchange of GDP to GTP in the protein. This exchange causes the activation of the G protein. In the following, the GTP-binding α-unit dissociated from the complex and transduces the signal to effector proteins, which release second messengers like cyclic AMP (cAMP), inositol-1,4,5-triphosphate (IP3), or diacylglycerol (DAG). These act on downstream signal cascades and on ion channels in order to induce an intracellular response (Lodish et al., 2000). Furthermore, stimulation of a GPCR was also shown to activate a second signaling circuit, which is mediated by G protein-coupled receptor kinases (GRK) and β-arrestins. Phosphorylation of the cytosolic terminus of the receptor by a GRK and subsequent binding of β-arrestins not only leads in desensitization by targeting the receptor complex into a coated pit, but β-arrestin-2 can also act as a scaffold for effectors of the mitogen activated protein kinase (MAPK) cascade and thereby relay a different type of signal in addition to the G protein-mediated response (McDonald et al., 2000). However, termination of a receptor signal is critically dependent on phosphorylation and endocytosis of the complex. Depending on the receptor type, the GPCR then either gets recycled to the membrane after dissociation of the ligand—like the endothelin A receptor (ETAR) (Paasche et al., 2005)—or it can get degraded and replenished by de novo synthesis—like the proteinase-activated receptor-2 (PAR-2) (Böhm et al., 1996). As pointed out above, a key role for the initial desensitization and internalization reaction is ascribed to β-arrestins, in this context. Moreover, GPCRs are associated with many diseases ranging from central nervous system disorders, including pain and depression, to metabolic disorders, such as diabetes or cancer (Drews, 2000). Due to their property to relay an exoplasmic signal with extreme specificity and to induce a defined intracellular response, they are seen as a well ‘druggable’ class of proteins. In fact, 40% of marketed drugs act on only 40 to 50 extensively characterized GPCRs (Eglen, 2005). These figures predict a high potential for the remainder of the class, half of it being uncharacterized ‘orphans’, meaning without a known endogenous ligand (Milligan, 2002). In order to find agonists or antagonists for GPCRs, several strategies are being applied in drug discovery. However, since GPCRs are not very abundant at the membrane—in general less than 10.000 proteins per cell (Ostrom and Insel, 2004)—most receptor studies must be over-expressed in eukaryotic cell lines. But apart from historically derived binding experiments or rational in silico drug design, functional assays constitute a relatively new class of assays that concentrate on functional responses in live cells and, hence, paved the way for the development of HCS systems.

Some concepts followed involve detection of second messenger molecules, whereas another widely used approach employs green fluorescent protein (GFP) as a fusion tag. A marker protein can be followed by means of fluorescence microscopy if translocation across the plasma membrane occurs as result of GPCR stimulation. In this regard, the GPCR-GFP fusion proteins permit detection and quantification of receptor endocytosis after stimulation (Tarasova et al., 1997). Disadvantages often associated with this technique are the labor-intensive establishment of the functional construct in recombinant cell lines, non-physiological binding as well as signaling and trafficking behavior.

Another type of assay makes use of a specific antibody-receptor interaction. Either the internalized receptor is detected at the membrane or in endosomes, respectively, by fluorescence labeled antibodies in fixed cells following compound addition. Alternatively, a translocation signal in live cells can be obtained when marking is carried out prior to compound addition. A disadvantage of this system is that large amounts of high quality, fluorescence-labeled antibody are needed. Furthermore, immunostaining after fixation is quite cumbersome and difficult to automate, whereas the approach in live cells mostly employs an N-terminal antigen-fusion tag, which can interfere with receptor conformation and ligand binding, especially when bound to an antibody (Eglen, 2005).

The object of the present invention is the establishment of a new generic principle to monitor the internalization of cell surface molecules of interest, in particular to monitor receptor-specific endocytosis. Such object is solved by the features of the independent claims; preferred embodiments are disclosed in the dependent claims.

The invention provides a method for monitoring a cell surface molecule and its potential internalisation into a cell on the surface of which is located said cell surface molecule of interest, comprising the steps of:
providing a sample carrier containing one or a plurality of cells which cell(s) possess a cell surface molecule of interest,
adding a detectable lectin or lectin derivative to the cell(s), which binds to the cell surface molecule of interest, and
monitoring the cell surface molecule of interest and its potential internalisation by detecting the lectin or lectin derivative.

Furthermore provided is a method for monitoring the internalisation of a cell surface molecule of interest into a cell on the surface of which is located said cell surface molecule of interest, comprising the steps of:
providing a sample carrier containing one or a plurality of cells which cell(s) possess a cell surface molecule of interest,
adding a detectable lectin or lectin derivative to the cell(s), which binds to diverse cell surface molecules including the cell surface molecule of interest,
stimulating the internalisation of the cell surface molecule of interest, and
monitoring the internalisation of the cell surface molecule of interest by detecting the lectin or lectin derivative.

The following paragraphs give an overview of the molecular background on which the invention is based.

Exo- and endocytosis are very dynamic processes of membrane locomotion that are of vital importance to the cell regarding lipid homeostasis, signal and substance transfer across the cell boundaries, and maintenance of cell polarization. Several mechanisms have been discovered for endocytosis that are responsible for the uptake of distinct classes of cargo into the cell: Clathrin-mediated endocytosis (CLAME), caveolae-mediated endocytosis (CAVME) and non-clathrin-non-caveolae-mediated endocytosis (NCNCME). In this regard, there is a variety of internalization pathways that co-exist, but still today distinct molecular makers could not be ascribed to all of them and the question remains, why the cell maintains this elaborate diversity (Mukherjee et al, 1997). Under consideration of this multitude of regulated routes, the fluid mosaic model of the cell membrane had to be revised since ordered areas of structures were found to exist within the membrane. They form functional units and thus were termed ‘microdomains’. One of these species that gained much attention are ‘lipid rafts’, or simply ‘rafts’, which constitute areas of clustered cholesterol and sphingolipids that float in the membrane bulk phase. Preferred localization of certain proteins to these rafts is seen as a result of partitioning equilibria between discrete lipid phases (Simon and Ikonen, 1997). In this regard, some receptors seem to be able to switch between partitioning modes in a stimulation-dependent manner (Ostrom and Insel, 2004; Le Roy and Wrana, 2005). Within rafts, small invaginations that are induced by binding of caveolin to the cytosolic side attract another subset of raft proteins in a selective manner. These are able to actively internalize via a dynamin-dependent mechanism. These immobile domains are termed ‘caveolae’. Even though simple raft domains were not yet found to recruit dynamin, a GTP-dependent protein that helps the vesicle to pinch off, they also carry out internalization independently of caveolin (Pelkmans and Helenius, 2002). However, both pathways have functional rafts and are therefore sensitive to cholesterol depletion. Since in most experimental cases a differentiation is impracticable, they are together referred to as raft-dependent endocytosis (RDE). Another type of microdomain is the clathrin-coated pit, also a small invagination in the membrane where clathrin covers the cytosolic face. In analogy to caveolae, only selected proteins may enter the pit—such as the constitutively internalizing transferrin receptor—whereas others are excluded (Gaidarov et al., 1999). Moreover, a crucial mechanism with respect to signal transmission by GPCRs and receptor tyrosine kinases (RTK) is that most of these proteins are only included upon activation, leading to a desensitization by subsequent removal from the cell surface (Santinti and Keen, 1996). Furthermore, a feedback mechanism exists that can reinforce CLAME when receptor induced signal transmission activates a downstream element (Wilde et al., 1999). Vesicle formation is also dynamin-dependent, but in contrast to CAVME, CLAME is a constitutive process.

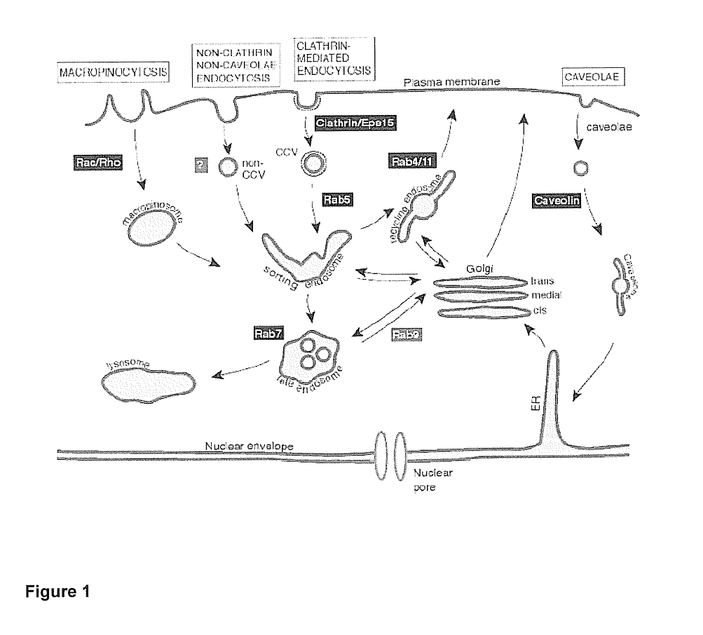

A brief overview of receptor trafficking shall be given with the focus on the best characterized clathrin-mediated pathway. After internalization, clathrin-coated vesicles disassemble their clathrin coat and fuse with the sorting endosome. This peripheral compartment exhibits a reduced pH of around 6 that promotes dissociation of receptor and ligand. Generally, the receptors are forwarded to the recycling compartment, having a pH of around 6.5, whereas most ligands are transferred to the more acidic compartment of the late endosome, then to the lysosome for degradation. Both endosomes were found to be rather spherical structures that locate to the perinuclear region. The recycling compartment was identified as a large, tubular structure either dispersed throughout the cytoplasm or arranged closely to the nucleus, depending on the cell type. From there, receptors get efficiently sorted and expelled in vesicles that return to the plasma membrane (Mukherjee et al, 1997). However, several interfaces with the pathway for delivery of de novo synthesized receptors, the macropinosome, and the RDE pathways exist. In this regard, the trans golgi network (TGN) represents a turntable organelle that is able to crosstalk with early, late, and recyling endosomes, also in a retrograde manner. Furthermore, it can act as an exit for delivery to the plasma membrane. In contrast, internalized caveolae were found to be destined for a separate compartment—the caveosome—the function of which remains to be clarified. The only connection to the endosomal system seems to lead across the endoplasmatic reticulum (ER), whereas the large macropinosome vesicles and non-clathrin-non-caveolae vesicles can gain access via the sorting endosome (Sieczkarski and Whittaker, 2002). Analytical identification of the compartments is done by tracing either marker proteins of each unit—like proteins of the Rab family—or well characterized cargo—such as the transferrin receptor (Sonnichsen et al., 2000). A model of the trafficking system and its markers are displayed in FIG. 1.

It can be summarized that a variety of trafficking routes coexist, which intersect and feed in one another, with the pathway through sorting and recycling endosome being most relevant for receptor recycling. Moreover, endosomes are characterized by distinct markers, shape, and spatial orientation within the cell.

Nearly all plasma-membrane proteins contain one or more glycosylations. These enable correct membrane delivery, modulate protein stability as well as binding affinity, and mediate cell-cell interaction (Lodish et al., 2000). Even though these principles have been recognized, the functional mechanisms that underlie glycan-based interactions have not been elucidated in detail. This is due to the enormous coding potential of oligomeric carbohydrate chains that surpasses nucleic acid-based and amino acid-based information and owing to the lack of analytics to deal with this diversity. Furthermore, oligomerization is non-template driven, which results in micro-heterogenic populations. Apparently, these impose another layer of complexity on the subject (Geyer and Geyer, 1998).

Protein glycosylation is a post-translational modification that is added as the proteins determined for secretion and membrane delivery move through the ER and TGN. Moreover, glycosylation was found to be species-, tissue-, cell-, and protein-specific and involves an elaborate set of carbohydrate processing enzymes. These are differentially expressed and reside in the cisternae of the respective organelles to generate a diverse array of glyco-patterns (Lottspeich and Zorbas, 1998). However, these patterns can be grouped to a limited set of basic structures. Firstly, protein glycosylations differ in the amino acid anchor and the core glycan: N-linked glycosylations exhibit a Man₃GlcNAc₂ core unit, which is attached to arginine residues within a fix signal sequon, whereas O-linked glycosylations are added to serine or threonine residues—apparently without a defined signal sequon—and contain a GaNAc core unit. N-linked glycans are mainly bi-, tri-, or tetra-antennary and can be further categorized into three classes with respect to the core extension. Briefly, high-mannose-type glycans exhibit predominately α-mannose units, complex-type glycans have GlcNAc substitutions with terminal sialic acid, and hybrid-type glycans have at least one branch of either of the two. O-linked glycans follow less pronounced rules and are generally rather short, containing only one to four residues (Lodish et al., 2000; Lottspeich and Zorbas, 1998). In addition to glycosylated proteins, sphingolipids, a sphingosine-based class of lipids, also features a glycan-carrying subgroup of similar complexity. Formerly being associated with structural properties only, now also functional roles for glycosphingolipids are getting emphasized (Futerman and Hannun, 2004). These new insights were brought into focus after the introduction of the lipid raft concept, which is a matter of intense research and debate. However, apart from the facts that sphingolipid assembly and glycosylation also occurs in the ER/TGN system and that several membrane bound glycosphingolipids are binding partners for viruses and bacterial toxins, few knowledge is currently available about this versatile group (Tsai et al., 2003; Sandvig and van Deurs, 2002). Due to a lack of interest and analytic methods, this field is still in its infancy.

It can be summarized that proteins and sphingolipids together display a diverse set of heterogeneous glycans on the cell surface. Structures show some categorical features, but only general concepts of glycosylation have been grasped, whereas the underlying functional interrelations have not been elucidated, yet.

Plant extracts were first reported to agglutinate erythrocytes in the late 19^{th} century, which subsequently led to the purification of these agglutinating proteins. Soon, they helped Paul Ehrlich to develop first concepts of immunology by using plant hemagglutins as model antigens and Karl Landsteiner to differentiate the three human blood groups. However, the carbohydrate-binding property—which was the basic principle of the reactions and implicated the presence of glycosylated structures on cell surface—was not recognized until the mid-20^{th} century (Sharon and Lis, 2004). Today, high purity preparations of the now called ‘lectins’ are commercially available, covering a broad spectrum of glyco-epitopes. Even though evidence for a physiologically relevant interaction of plant lectins with mammalian cells is still lacking, it did not hamper the utilization of these carbohydrate-specific probes for histological applications in studies of animal development and disease, as well as for in vitro methods in the analysis of glycoconjugates (Danguy et al., 1998; Geyer and Geyer, 1998).

On the other hand, endogenous carbohydrate recognition events also must have specific proteins in the animal cell. However, since the majority of them are membrane-bound or additionally localized to intracellular organelles, their identification and characterization was significantly impaired. Moreover, the state of glycan research mentioned, did not give a strong impetus to push the search for endogenous lectins, albeit genome projects now offer data for a rational approach to discover these proteins by means of bioinformatics (Gabius, 1997).

Another set of lectins is of bacterial and viral origin. In contrast to plant lectins, they have a distinct role in mediating entry of the pathogen into the eukaryotic cell. As mentioned earlier, infection mechanisms generally involve uptake by endocytosis. This fact pinpoints the role of glycosylation as an efficient handle to endocytic processes.

In this regard, plant lectins have been used as carbohydrate specific probes ever since their discovery. A broad set with distinct binding properties is commercially available. Even though research on animal, bacterial, and viral lectins is still behind, the role of the latter two in host invasion through endocytosis hints at an option for a glycosylation-targeting endocytosis assay.

In the following, preferred embodiments of the present invention are disclosed.

It is preferred that the internalisation of the cell surface molecule of interest is stimulated by adding a chemical compound to the cellular sample. Such chemical compound may be a compound under investigation within a drug discovery campaign (including primary and secondary screening processes) to identify specifically those compounds which stimulate the internalisation of a specific cellular surface molecule of interest, i.e. agonists. The method may also be used to identify antagonists or other types of modulators influencing the internalisation of the cellular surface molecule of interest. Within the identification of antagonists, it is preferred to add in a first step the presumed antagonist compound to the cellular sample under investigation. Thereafter, in a second step (or alternatively simultaneously with the first step) a compound known to induce the internalisation of a specific cellular surface molecule of interest is added. If, after addition of such inducer compound, no or diminished internalisation of the cellular surface molecule takes place, the presumed antagonist compound is indeed an antagonist.

Preferably, the cell surface molecule of interest comprises a protein or a lipid molecule. Such protein or lipid molecule comprises a lectin or lectin derivative binding site, which binding site preferably comprises a glycosylated protein or lipid moiety. In particular, the protein molecule is a cell surface receptor. Such cell surface receptor may e.g. be a G-protein coupled receptor, a receptor tyrosine kinase, an ion channel, a cell adhesion molecule, a hormone receptor, a cytokine receptor, a chemokine receptor, a growth factor receptor, a neurotransmitter receptor, a lipoprotein receptor, a vitamin receptor, a viral binding receptor, a bacterial-interacting receptor, an antibody receptor, or a complement-binding receptor. The aforementioned lipid molecule may preferably be a glycolipid, a glycoglycerolipid, a glycoshingolipid, a glycophosphatidylinositol, a psychosine, a glycoglycerolipid, a ceramide, a monoglycosylceramide, a diosylceramide, a ganglioside, a glycuronosphingolipid, a sulfoglycoshingolipid, or a phosphonoglycosphingolipid.

Preferably, the cell surface molecule of interest is a protein which is over-expressed in the cell. However, it is also possible to use a wild-type cell comprising the cell surface molecule of interest preferably in a high amount. The use of the aforementioned cell types is particularly preferred in terms of establishing a good signal-to-noise ratio when monitoring the internalisation of the cell surface molecule of interest.

In a preferred embodiment, the detectable lectin or lectin derivative is luminescently, preferably fluorescently, or radioactively labelled. It is particularly advantageous to use a fluorescently labelled lectin or derivative thereof. The detectable lectin or lectin derivative may be monitored by optical methods such as microscopy, preferably automated microscopy; automated fluorescence reader for the conductance of the method of the present invention are readily available on the commercial market. It is particularly preferred to use confocal microscopy due to its high resolution capability.

In particular, in the conductance of the method according to the present invention a medium comprising a background reducing agent, in particular insulin, is added. Such background reducing agent compresses preferably non-receptor mediated fluid phase endocytosis processes.

The detection of the internalisation of the cell surface molecule of interest to which a luminescently, preferably fluorescently, labeled lectin or lectin derivative is bound may be performed by measuring a decrease of luminescence, preferably fluorescence, on the cell surface membrane. In particular, the degree of internalisation may be determined by comparing the amount of detectable lectin or lectin derivative bound to the cell surface before and after stimulation of the internalisation process. Alternatively, the detection of the internalisation of the cell surface molecule of interest to which a luminescently, preferably fluorescently, labeled lectin or lectin derivative is bound is performed by measuring an increase of luminescence, preferably fluorescence, within the cell, in particular within cytoplasmic compartments such as endosomes. In particular, the degree of internalisation may be determined by comparing the amount of detectable lectin or lectin derivative inside the cell, preferably inside the cytoplasm and/or nucleus, before and after stimulation of the internalisation process. In another embodiment, the detection of the cell surface molecule of interest to which a radioactively labelled lectin or lectin derivative is bound is performed by measuring a decrease of radioactivity on the cell surface membrane and/or an increase of radioactivity within the cell, in particular within cytoplasmic compartments such as endosomes.

It is preferred to determine the area of cytoplasmic compartments, the fluorescence intensity within cytoplasmatic compartments, and/or the number of cytoplasmic compartments comprising detectable (e.g. fluorescently labelled) lectin or lectin derivative as a measure for the internalisation.

The method according to the present invention may particularly be used for identifying compounds that induce or inhibit the internalisation of cell surface molecules. In particular, it may be used in drug discovery and drug development.

### EXPERIMENTS

The invention will be further described in more detail in experiments and figures below. Within this context, the following abbreviations are used:

μg microgram
μl microliter
μM micromolar
Abs absorbance
Avidin-HRP avidin-horseredish peroxidase conjugate
BCA bichinonic acid
BSA bovine serum albumin
cAMP cyclic AMP
CAVME caveolae-mediated ednocytosis
CLAME clathrin-mediated endocytosis
DAG diacylglycerol
ECL enhanced chemi-luminescence
ER endoplasmatic reticulum
ETAR wildtype Endothelin A Receptor
ETAR-GFP Endothelin A receptor-green fluorescent protein fusion construct
g gramm
GFP green fluorescent protein
GPCR G protein coupled receptor
GRK G protein coupled receptor kinases
h hour
HCS high content screening
HRP horseredish peroxidase
IP3 inositol-1,4,5-triphosphate
MAPK mitogen activated protein kinase
min minutes
ml milliliter
mM millimolar
ms millisecond
MS modified stimulation
NCNCME non-clathrin-non-caveolae-mediated endocytosis
ng nanogram
nM nanomolar
NS no stimulation
OS original stimulation
PAR-2 proteinase-activated receptor 2
PBS phosphate buffered saline
PFA paraformaldehyde
PI3K phosphatidylinositol 3 kinase
rcf relative centrifugal force
RDE raft-dependent endocytosis
RNAi RNA interference
RT room temperature
RTK receptor tyrosine kinase
s second
SR spot ratio
TGN trans golgi network
TRITC tetramethylrhodamine isothiocyanate
U2OS human osteosarcoma cell line

### Material and Methods

### 1. Sources of Materials

In the experiments conducted according to the present invention, all chemicals (other than special reagents mentioned below) were purchased from Sigma-Aldrich Chemie (München, Germany) and Bio-Rad (München, Germany) at the highest purity available. All organic solvents and acids were purchased from Sigma-Aldrich Chemie or Merck (Darmstadt, Germany). Bi-distilled water (ddH₂0) with a maximum conductivity of 0.055 μS/cm was used from the company's internal Reinstwassersystem UV-Plus (SG Wasseraufbereitung and Regeneration, Hamburg, Germany) water system.

The sources of special reagents were as follows: Biotin labeled lectins (RLK 3200) and tetramethylrhodamine isothiocyanate (TRITC) labeled lectins (BK 2000) were purchased from Vector Labs (Burlingame, U.S.A) as sampler kits containing seven labeled plant lectins: Griffonia (Bandeiraea) simplicifolia lectin (GSL I), Pisum sativum agglutin (PSA), Lens culinaris agglutin (LCA), Phaseolus vulgaris erythroagglutin (PHA-E), Phaseolus vulgaris leucoagglutin (PHA-L), Sophora japonica agglutin (SJA), and succinylated Triticum vulgaris (wheat germ) agglutin (sWGA). Avidin-horseredish peroxidase conjugate (Avidin-HRP) (A115) was purchased from Boston Biochem (Cambridge, U.S.A.), mouse anti-endothelin A receptor monoclonal antibody (612629) was purchased from BD Bioscience (Heidelberg, Germany), mouse anti-protease activated receptor 2 monoclonal antibody SAM 11 (sc-13504), as well as goat anti-mouse antibody-HRP conjugate (sc-2030) were from Santa Cruz Biotechnology Inc. (Santa Cruz, U.S.A.). Human endothelin 1 (E7764), porcine insulin (I6634), and protease inhibitor cocktail (P8340) were obtained from Sigma-Aldrich and human protease activated receptor 2 agonist peptide from Bachem (Weil am Rhein, Germany). Human holo-transferrin-Alexa Fluor® 488 conjugate (T-13342) and Hoechst 33342 (H-3570) were from Molecular Probes—Invitrogen (Karlsruhe, Germany). DRAQ5™ (BOS-889-001-R200 via Axxora, Grünberg, Germany) was obtained from Biostatus (Leicestershire, U.K.). ECL Plus western blot detection kit (RPN2132), Hybond ECL nitrocellulose blotting membranes (RPN2020D), and Hyperfilm (RPN3103K) were obtained from Amersham BS (Uppsala, Sweden). The micro BCA protein assay kit (23235) was from Pierce (Rockford, U.S.A.) and the SDS-PAGE standard broad range marker (161-0317) from Bio-Rad (Munchen, Germany).

The sources of cell culture reagents were as follows: Standard cell culture ware, such as T75 and T175 flasks and pipetting materials, were from Greiner Bio-One (Frickenhausen, Germany) and Corning B.V. (Schiphol-Rijk, Netherlands) respectively. Plastic bottom 96-well ViewPlates (6005182) were obtained from Packard—PerkinElmer (Boston, U.S.A.). Phosphate buffered saline (PBS) for cell culture (D8537), trypsin solution (T3924), and foetal calf serum (FCS) were purchased from Sigma and Versene EDTA solution (BE17-711E) from Cambrex (Baltimore, U.S.A.) Hank's balanced salt solution (HBSS) (14065-049), basal media for eukaryotic cell culture, and antibiotics were all purchased from Gibco-Invitrogen (Karlsruhe, Germany) as listed in Table 1.

The sources of cell lines were as follows: U2OS human osteosarcoma cell line stably expressing a functional endothelin A receptor-green fluorescent protein-fusion construct (ETAR-GFP) were prepared in-house; this also applies to CHO-K1 cell line clone #19 stably expressing a wild type endothelin A receptor (ETAR) and a CHO-K1 cell line clone #04 stably expressing a functional wild type protease activated receptor type 2 (PAR-2). Table 1 lists media formulations used for culturing these cells.

### 2. Buffers and Solutions

The following buffers and solutions were prepared as stock solutions for routine protocols.

Assay Medium
Hank's Balanced Salt Solution (HBSS)

| | |
|---|---|
| HEPES | 20 mM |
| D-Glucose | 30 mM |

Hypotonic Lysis Buffer pH 7.4

| | |
|---|---|
| Tris-HCl | 10 mM |
| MgSO₄ | 1 mM and |
| EDTA | 0.5 mM |
| Protease Inhibitor | 100 μl/10⁸ cells |
| Cocktail | |

3% PFA Solution pH 7.4

| | |
|---|---|
| Paraformaldehyd | 3% (w/v) |
| MgCl2 | 100 μM |
| CaCl2 | 100 μM |
| 0.45 μm sterile filtered | |

SDS-PAGE - Gel Solutions

| | |
|---|---|
| Tris-HCL pH 6.8 | 0.75 M |
| Tris-HCL pH 8.8 | 0.75 M |
| SDS | 10% (w/v) |

SDS PAGE - Loading Buffer (2×)

| Tris-HCl | 62.5 mM |
|---|---|
| SDS | 2.3% (w/v) |
| Glycerin | 10% (w/v) |
| β-mercapto-ethanol | 5% (v/v) |
| Bromphenol-blue | 0.01% (v/v) |
| Pyronin G | 0.01% (v/v) |
| Acrylamid/N,N′- | 30/0.8% (w/v) |
| methylene-bis- | |
| acrylamide | |
| APS | 10% (w/v) |
| (Prepared freshly) | |
| TEMED | 100% |

SDS PAGE Running Buffer (Laemmli)

| | |
|---|---|
| Tris | 25 mM |
| Glycine | 192 mM |
| SDS | 0.1% (w/v) |

Blotting - Cathode Buffer (CB)

| | |
|---|---|
| Tris-HCL pH 8.4 | 25 mM |
| Methanol | 20% (v/v) |
| ε-aminocaproic acid | 40 mM |
| SDS | 0.1% (w/v) |

Blotting - Anode Buffer (AB) I

| | |
|---|---|
| Tris-HCL pH 10 | 300 mM |
| Methanol | 10% (v/v) |

Blotting - Anode Buffer (AB) II

| | |
|---|---|
| Tris-HCL pH 9.4 | 25 mM |
| Methanol | 10% (v/v) |

Coomassie Staining Solution

| | | |
|---|---|---|
| Methanol | 46% | (v/v) |
| Acetic acid | 8% | (v/v) |
| Coomassie Brilliant | 0.2% | (w/v) |
| Blue R250 | | |

Destaining Solution

| | | |
|---|---|---|
| Methanol | 20% | (v/v) |
| Acetic acid | 10% | (v/v) |

Phosphate Buffered Saline (PBS) pH 7.4

| | |
|---|---|
| NaCl | 150 mM |
| KCl | 2.7 mM |
| Na₂HPO₄*2H₂O | 6.5 mM |
| KH₂PO₄ | 1.5 mM |

PBS-T

| | |
|---|---|
| PBS | 99.9% (v/v) |
| Tween 20 | 0.1% (v/v) |

### 3. Instrumentation, Procedures and Software

Cell culture procedures were conducted under a HERASafe® KS Safety Cabinet Class II and cell incubations were done in a HERAcell® CO₂ Incubator, Heraeus Instruments (Langenselbold, Germany) at 37° C., 5% CO₂ and 95% humidity.

All centrifugation procedures were performed with fixed angle rotors in either a Biofuge pico, a Megafuge 1.0R (both Heraeus Instruments) or an ultra-centrifuge Sorvall Discovery 90SE (Langenselbold, Germany), as noted in the respective method.

For cell homogenization a 5 ml potter from Satorius-Braun Biotech (Melsungen, Germany) was used.

Spectrometric analysis was conducted with a Tecan Safire™ Microplate Reader (Mannedorf, Switzerland), and data were processed with the supplied XFluor™ Data Evaluation Software.

For polyacrylamide gel electrophoresis and subsequent blotting procedures the Mini-Protean™ 3 system, the Trans-Blot SD semi-dry transfer cell, and the corresponding power stations Power Pac 200 and 300 (all Bio-Rad, Munchen, Germany) were used. For downstream processing of western blots the Enhanced Chemiluminescence System (ECL) from GE Healthcare was used employing a Hyperprocessor™ Automatic Film Processor (also GE Healthcare) for film development. Films were scanned using a Mustek 1200TA Scanner (Neuss, Germany).

Olympus CK30 microscopes (Melville, U.S.A.) were used for routine laboratory microscopy. Epifluorescence microscopy was conducted using an Olympus XI70 fluorescence microscope, which was equipped with 3 objective lenses for 10-fold, 20-fold, and 40-fold magnification and a filter set with four filters: U-MNU, U-MSWB (both Olympus), U-N41007, and U-MNIBA (both Chroma, Rockingham, U.S.A.). These covered the excitation spectrum from 250 nm to 500 nm and the respective emission windows for widely used chromophores. Image recording and processing was accomplished with a standard 1.3 Megapixel CCD camera F-View and the AnalySIS® image analysis software (both Soft Imaging System, Munster, Germany). Automated confocal fluorescence microscopy was conducted using an Opera™ QEHS microplate imaging reader, software version 1.7.1, and data evaluation was carried out with the respective Acapella™ 1.0 high content data analysis software, both from Evotec Technologies (Hamburg, Germany). The instrument was equipped with interchangeable water objective lenses for 10-fold, 20-fold, and 40-fold magnification, four excitation lasers (405 nm, 488 nm, 532 nm, and 635 nm) and a high-pressure Xenon epifluorescence UV lamp. Selectable filter sets allowed for simultaneous fluorescence detection with up to three CCD cameras. The Acapella™ software comprised a library of cell recognition scripts, which could be individually combined. Furthermore, script related parameters were tunable to optimize the detection algorithm for a given fluorescence signal.

### 4. Routine Cell Culture

Cells were continuously kept in culture in T75 flasks by incubation at 37° C. and 5% CO₂ and 95% humidity. Splitting was carried out at 80-100% confluence by washing with 5 ml PBS, applying 1 ml trypsin solution onto the cells for two to three minutes, and taking them up in 9 ml new medium. Cells were seeded according to the desired splitting ratio. Splitting ratios routinely used were 1:2 to 1:20. Counting of cells was accomplished with a Neubauer counting chamber.

### 5. Lectin Mediated Fluorescence Labeling

Lectin labeling of fixed cells was conducted as follows: To detach confluent cells in order to prepare experiments for internalization studies, cells were always treated with EDTA solution instead of trypsin solution. For all seeding, washing, and labeling steps in 96-well plates a working volume of 100 μl per well was assessed. Cells were seeded out into a 96-well plate at different concentrations. U2OS/ETAR-GFP suspensions were adjusted to yield 2*10⁴ cells/well, both of the CHO cell lines must be at 2.5*10⁴ cells/well. Cells became confluent after 24 hours of incubation. In the following, they were washed with phosphate buffered saline (PBS) and fixed with 3% PFA for 20 minutes at RT. Then, cells were washed again with PBS and incubated in 10 nM and 20 nM solutions of each TRITC-lectin candidate in PBS, respectively. Again, cells were washed with PBS to remove excess lectin, and wells were refilled with PBS for analysis. For specificity controls, lectin solutions were incubated for 30 minutes prior to the application with an excess of the monosaccharide that represents the respective glyco-epitope. Table 2 displays the employed lectins, their glyco-epitopes, and the corresponding inhibitory monosaccharides.

Lectin labeling of live cells was conducted as follows: Cells were seeded as described above. After 24 hours cells were washed once with PBS and incubated in the incubator for another 2 hours in the respective standard medium without serum supplement to deprive them of serum factor mediated stimuli. This step was termed starvation period. Subsequently, cells were washed gently with PBS and incubated with 37° C. tempered assay medium comprised of Hank's Balanced Salt Solution (HBSS) pH 7.4, containing 20 mM HEPES and 30 mM D-glucose. The plate was left at RT for 30 minutes to slowly level its temperature and, thus, avoid capturing of temperature drop induced membrane dynamics. This period was termed leveling period. Then, assay medium was withdrawn and wells refilled with TRITC-lectin solutions at RT to start cell labeling. Several lectin concentrations in the low nano-molar range were tested. Molar concentrations were calculated from the data provided by Vector Labs. The plate was placed on a rotating shaker at low revolutions for 10 minutes while incubating. The start of the lectin incubation is referred to as assay starting point with t=0 min. After the 10 min incubation period the cells were gently washed with PBS and wells refilled with assay buffer at RT. Instantly thereafter, fluorescence signals were monitored predominately using the epifluorescence microscope.

For serum supplements in live cell labeling, starvation was carried out in Opti-MEM medium (11058; Gibco—Invitrogen, Karlsruhe, Germany). The following labeling and chase incubations were conducted in assay medium with a 20 nM insulin supplement.

With regard to the internalization pathway analysis in U2OS/ETAR-GFP cells, RDE was inhibited by including 5 mM β-methyl-cyclodextrin (CD) in the leveling medium. The following labeling and chase incubations were conducted in standard assay medium. For inhibition of clathrin mediated endocytosis cells were subjected to hypotonic shock by incubating 5 minutes at 37° C. in a 1:1 starvation medium-ddH₂O mixture and then were transferred into a potassium (K⁺) depleted assay medium for temperature leveling. The following labeling and chase incubations were also conducted in K⁺ depleted assay medium, which was prepared by substituting sodium salts for all potassium salts in HBSS. Inhibition of both pathways was achieved by combining both methods and including 5 mM CD in the leveling medium for CLAME inhibition. For CLAME controls, 5 μg/ml Alexa Fluor 488 conjugated transferrin was included during the labeling period for all inhibited pathway scenarios. Protocol procedures were conducted as described, however, lectins were omitted in the labeling step. For lipid raft-dependent pathways there were no control markers available.

With regard to lectin labeling of live cells with receptor target stimulation, lyophilized PAR-2 peptide was resuspended in 20 mM HEPES with 0.1% BSA at 4 mM. Lyophilized endothelin 1 was solubilized in dimethylsulfoxide (DMSO) at 40 μM. The live cell labeling protocol was applied as described above. Additionally, endothelin and PAR-2 agonist peptide were added to the assay medium employed after the lectin labeling step at concentrations of 40 nM and 100 μM, respectively. For controls, same amount of resuspension liquid without agonist was supplemented. Instantly, fluorescence signals were monitored.

With regard to lectin labeling of live cells with modified stimulation, in the protocol for receptor targeted stimulation all steps from the lectin labeling step onwards were conducted in the presence of the given agonist. Furthermore, cells were fixed with PFA as in 2.5.1 at t=20 minutes and t=30 minutes. Fixed cells were stained by incubation for 1 hour with 5 nM DRAQ5 in assay medium. Fluorescence microscopy was carried out on the epifluorescence microscope and on the Opera confocal imager for detailed signal quantification four days after the experiment.

The following microscopy and image analysis techniques were employed. When utilizing the epifluorescence microscope magnifications were adjusted according to the purpose of observation, e.g. single cell vs. cell collective. Concerning the filters U-MNU was used for Hoechst, U-MSWB for GFP, and U-N41007 for TRITC detection. Hoechst images were taken at 50 ms exposure time, GFP images at 100 to 200 ms and TRITC images needed 500 to 1000 ms. Overlay images were assembled with AnalySIS and tuned in color intensity to obtain the desired color contrast. Images taken to compare two experimental conditions, e.g. control vs. stimulus, featured identical exposure parameters. If possible, well areas were chosen that reflect similar states of confluence. Utilizing the Opera confocal fluorescence imager, the instrument was calibrated running standard methods for skew cropping (spatial camera alignments) and camera intensity normalization. Table 3 lists the parameters used for imaging TRITC and DRAQ5™ signals.

### 6. Lectin and Western Blotting for In Vitro Binding Analysis

Membrane preparation was conducted as follows: Cells were cultured in five T175 flasks until confluency. Confluent cells were detached by applying 3 ml of EDTA solution onto the PBS washed cells. Following a 20-minute incubation at 37° C. and 5% CO₂, cells were resuspended in 12 ml of starvation medium (standard medium without FCS), counted, and centrifuged in a Heraeus Megafuge 1.0R at 133 rcf for 4 minutes. All subsequent steps were carried out on ice and with ice cold solutions. After unification of all pellet fractions, hypotonic lysis buffer was added to the cells at a ratio of 1 ml per 5*10⁷ cells. Additionally, protease inhibitor cocktail was added to 50 μl per 5*10⁷ cells. Cells were homogenized with 30 strokes in a 5 ml potter on ice, centrifuged at 4° C. and 917 rcf for 10 minutes, and the supernatant was stored in an ultracentrifuge tube on ice. The pellet fraction was homogenized and centrifuged once again as described above. The supernatants of both procedures were unified and centrifuged at 4° C. and 100,000 rcf for 45 minutes in a Sorvall ultra-centrifuge. The supernatant of this centrifugation procedure was withdrawn and the pellet dissolved in 0.5 ml or 1.0 ml modified SDS PAGE loading buffer containing Tris-HCl, SDS, glycerol, but no β-mercapto-ethanol or dyes and stored in small aliquots of 50 μl at −20° C.

The determination of protein content was conducted as follows: Protein concentrations were determined employing a bichinonic acid (BCA)—based assay kit from Pierce and following the product description provided with the kit. Briefly, assay reagent was mixed 1:1 with a standard dilution series of BSA (≦200 μg/ml) and a dilution series of membrane samples, respectively, to yield 300 μl per well in a microtiter plate. Preparations were executed in duplicates. The plate was left at 37° C. for 1.5 hours and subsequently read at λ=562 nm with a Tecan Safire™ spectrometer. Membrane sample absorbance values between 0.4 and 1.0 were considered for determination of protein content based on a linear regression of averaged values of the BSA dilution. Sample protein content was regarded as BSA equivalent concerning the absorption coefficient and, thus, was converted without a correction factor.

SDS-Page of membrane samples was conducted as follows: Discontinuous 12% polyacrylamide gels were prepared according to a standard protocol. Broad range marker and 25 μg membrane protein were mixed 1:1 with loading buffer and loaded five times onto a mini gel in alternating order. The gels were run at U=100 V for the first 20 minutes. Subsequently, voltage was increased to U=150 V. The run was aborted as soon as the pyronin G band reached the end of the gel. For coomassie staining, gels were put in coomassie staining solution on a shaker at low revolutions for 45 min. The staining solution was removed and the gel incubated with destaining solution for one to two hours, exchanging the solution several times until the desired contrast was obtained.

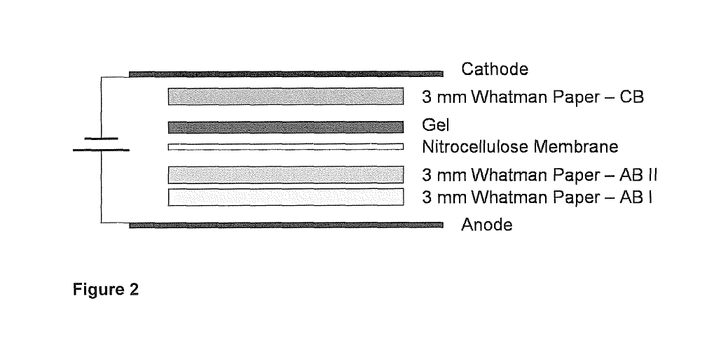

Blotting of membrane samples was conducted as follows: For blotting, 3 mm packs of Whatman papers were extensively soaked in one of the diverging blotting buffers, AB I, AB II, and CB, and a pile was assembled in a semi-dry Trans-Blot 3D system from Bio-Rad as depicted in the sketch of FIG. 2. Hybond nitrocellulose membrane was submerged in AB II prior to assembly. To smooth out any air bubbles from the pile, a Pasteur pipette was rolled several times over the top layer. The apparatus was closed and ran at U=25 V for 45 minutes. Then, the pile was disassembled, the membrane submerged in Ponceau S solution and gently swayed for 2 minutes. For destaining, ddH₂O was substituted for the staining solution, and the membrane was briefly rinsed twice. After drying under ambient conditions, the marker bands were marked with a ballpoint-pen. The membrane was cut into slices each displaying one marker and one sample lane.

Probing of membrane samples was conducted as follows: For classical western blotting, membranes were blocked in PBS containing 5% low fat dried milk powder. They were placed on a rotating shaker at low revolutions for 1 hour. Subsequently, membranes were sealed in plastic bags with blocking solution additionally containing 1 μg/ml anti-PAR-2 antibody (1:400) or 0.5 μg/ml of anti-ETAR antibody (1:500), depending on the respective recombinant cell type used for the sample. The bags were placed on a shaker at medium revolutions for 1 hour. Following the primary antibody step, membranes were washed four times with increasing volumes of PBS-T. For the secondary antibody step, membranes were incubated in blocking buffer with 160 ng/ml goat anti-mouse-antibody-HRP (1:2500) for 1 hour at low revolutions. The washing was repeated with increasing volume of PBS-T. A final wash with pure PBS was appended.

For lectin blotting, the blocking buffer was changed to 10% polyvinylpyrrolidone in PBS, which was also used in the detection incubations. Washing steps were not modified. However, biotinylated lectins were used as primary detection reagents at a concentration of 5 μg/ml and avidin-HRP fusion protein as secondary detection reagent at a concentration of 5 ng/ml (1:100,000). For specificity controls of secondary reagents, primary reagents were omitted. Enhanced chemiluminescence (ECL) mediated detection of HRP-conjugates was identical in both procedures. It was carried out according to the protocol of ECL plus kit from Amersham. Briefly, ECL plus reagent was mixed and pipetted onto the membranes, incubated for 5 minutes, and poured off the membrane strips. These were carefully dried on both faces with kim-wipes and placed under transparent plastic foil. Films were exposed to the luminescent membranes for various periods from 20 seconds to 10 minutes and subsequently developed and scanned for documentation.

### Experimental Results

### 1. Lectin Mediated Fluorescence Labeling

As outlined above, the present invention provides a new generic assay principle for monitoring the internalization of cell surface molecules of interest into a cell on the surface of which is located said cell surface molecule of interest; in particular, the present invention provides a new generic assay principle for the monitoring of receptor-specific endocytosis. The rationale behind this preferred embodiment of the present invention was to label glycosylated cell surface components including a receptor of interest with fluorescent lectins and to monitor its internalization upon stimulation. Using fluorescence microscopy, one is able to assess and quantify the reaction. In this regard, experiments were designed that allowed for a stepwise investigation of the underlying principles of lectin-cell interaction.

### 1.1 Lectin Labeling of Fixed Cells

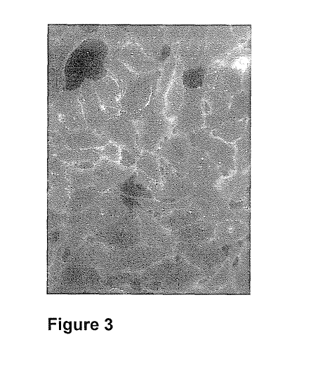

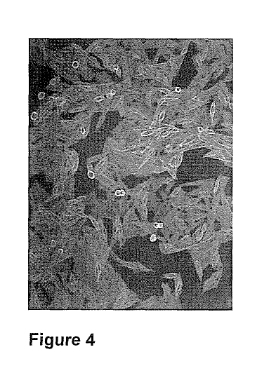

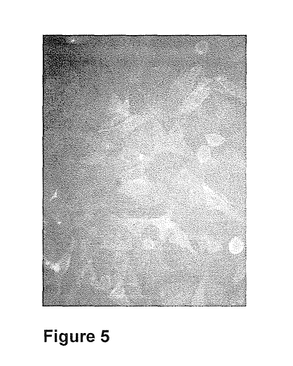

In order to investigate lectin binding to the cell surface in a first approach, fixed cells were used to obtain a freeze image devoid of interference by cellular dynamics. Herefore, cells were fixed by PFA and tested with a panel of seven TRITC-labeled plant lectins. In the experiments, three different cell lines were used: a U2OS/ETAR-GFP cell line, and two different CHO cell lines, one carrying the human endothelin A receptor (ETAR) the other the human proteinase-activated receptor-2 (PAR-2). With U2OS/ETAR-GFP cells, extensive fluorescence was observed at the cell surface using sWGA, PSA, LCA, PHA-E, and PHA-L, allowing for recognition of each single cell in the collective. FIG. 3 depicts an example. In this regard, PHA-E and PHA-L caused very bright signals at cell-cell contact areas. GSL-I labeling resulted in a very faint signal covering only some single cells, and SJA exhibited only several little, spot-like signals on each cell. The labeling of CHO/ETAR cells caused similar signals compared to U2OS cells using PHA-L, PHA-E, LCA, and sWGA, albeit, free cell boundaries but not cell-cell contact areas were intensively fluorescing with the PHA lectins, as displayed in FIG. 4. Examining CHO/PAR-2 cells, only PHA-L, PHA-E, and sWGA caused signals clearly marking individual cells. Highlighting of distinct cell areas by PHA lectins was diminished. Moreover, GSL-I, LCA, and PSA gave signal patterns similar to the aforementioned, but these exhibited reduced intensity with a high background. This kind of signal is depicted in FIG. 5. Table 4 gives an overview of the type of staining obtained with the lectins on the individual cell lines. In order to test whether lectin staining was a specific binding process, labeling experiments were carried out, where the monosaccharide that represents the respective glyco-epitope of the lectin was added in excess. It was expected that competition should prevent the lectin from binding to the cell. In these studies conducted with U2OS cells, none of the lectins but SJA, PHA-E, and PHA-L exhibited a specific fluorescent signal in the presence of the competing monosaccharide. However, signals from PHA-L, PHA-E, and SJA were reduced.

In another series of experiments, staining efficiency was tested at several concentrations to estimate binding capacity of the cells. It was found that fluorescence intensity seemed to correlate with the applied concentration of lectin between 10 nM and 20 nM. Best signal yields were obtained at the highest lectin concentrations.

Summarizing the results obtained with fixed cells, it can be stated that a glycosylation-specific staining could be achieved with the individual lectins, albeit, labeling efficiency was found to be dependent on the lectin candidate and the cell type employed. Differences in labeling performance were also found comparing the two CHO cells types. Moreover, high lectin concentrations tested led to a better signal yield.

### 1.2 Lectin Labeling of Live Cells

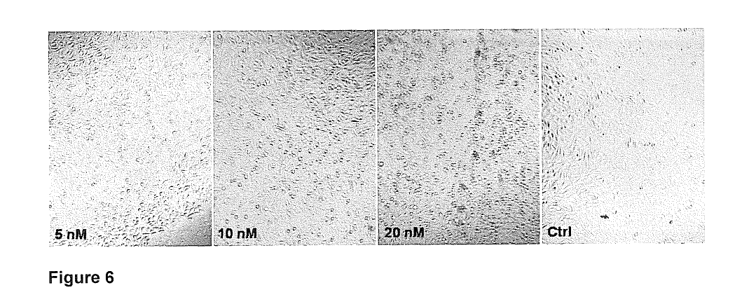

In addition to the above described experiments on fixed cells, a series of experiments for live cell labeling were set up as the next step. In principle, the experimental procedure for the live cell labeling was adopted from the fixed cell approach. It followed the scheme of a pulse-chase design to investigate the behavior of fluorescent lectins without prior fixation. The protocol employed had been iteratively optimized in order to meet the demands on nutrients, starvation periods, pH level, osmolarity, incubation intervals, and temperature adjustment. In a first approach, lectin concentrations where used as before. However, it soon became evident that the lectin incubations caused an irritation to the normal morphology of the cells. Cells became rounder and less tense at their cell boundaries already at 5 nM lectin. Increasing the concentration to 20 nM, a significant number of cells detached from the collective, but remained adhered to the confluent layer. FIG. 6 illustrates this tendency for U2OS cells labeled with SJA. Since high lectin concentrations interfered with the normal physiology, subsequent experiments were conducted at concentrations of 10 nM at maximum.

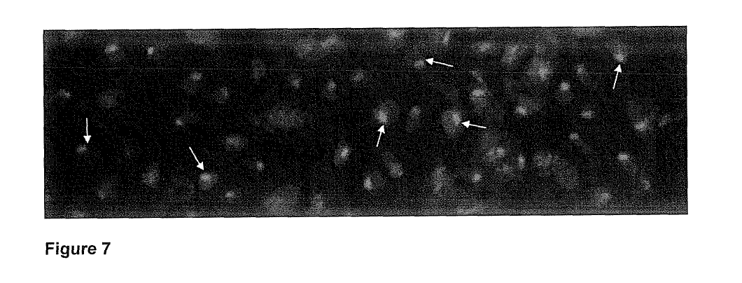

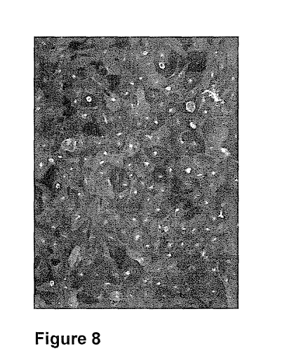

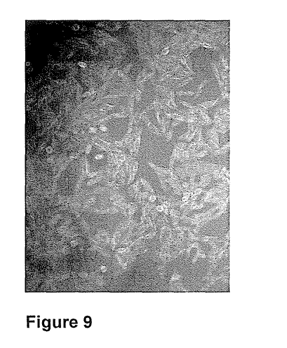

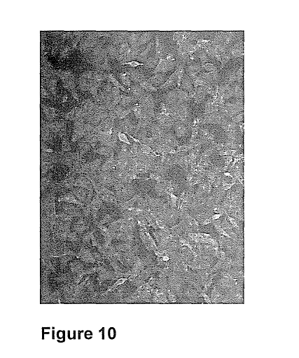

Comparing live cell to fixed cell labeling, it should be taken into account that a signal development taking place during the initial labeling step could not be assessed. Therefore, all observations represented a signal state t≧10 min. In this series of experiments, the same cell lines were used as in the previous. With these cells, some general observations were made during microscopy, which shall be pointed out here. Firstly, the first fluorescence signal captured, generally at t=12 min, deviated already from the freeze image observed with the fixed cells. Nonetheless, key characteristics of the fixed cell staining, like the signal-to-noise ratio, signal intensity, or distinct marking patterns on the cells, could clearly be recognized in the live cell image. Secondly, a very striking feature was the emergence of a spot-like accumulation of fluorescence in proximity to the nucleus, as depicted in the overlay image of lectin and nucleus signals in FIG. 7. The processed image furthermore revealed that the majority of nuclei exhibited a curved shape with an inversion towards the spot signal. Moreover, from the microscopic observations a distinctive scheme for the development of this lectin signal over a period of several hours could be recognized. It is summarized in Table 5 and involves initial sequestering of signals at the membrane, formation of lager spots at more centered positions and, finally, the unification of signals in the perinuclear region. Over the whole process, homogeneous fluorescence at the cell membrane decreases. Most lectins investigated followed this scheme to a certain extend. Thirdly, when signals of different lectin concentrations were compared, in almost all cases a reinforcement of the dynamic signal was found with increased concentration, whereas a reduction caused a tendency towards the stationary type of signal observed with fixed cells. In contrast to these tendencies, which were deduced from common observations with all cell lines, very specific findings were also made for the individual cell line. Concerning U2OS cells, all lectins but GSL-I and SJA displayed a signal course as described in Table 5. However, also GSL-I and SJA, that exhibited only marginal signals on fixed cells, displayed little spots over the whole cell surface. SJA developed a stronger signal in the form of medium sized, scattered spots, GSL-I signal points were significantly smaller and less. Therefore, both lectins showed a distinct transformation but did not undergo single spot formation. A typical spot signal observed with sWGA is depicted in FIG. 8. With CHO/ETAR cells, only lectins that produced a clear labeling pattern on fixed cells were investigated. sWGA went through the signal course outlined in a similar manner compared to U2OS cells, albeit, single spot formation continued fairly slowly and homogeneous cell surface fluorescence was maintained at a higher level over the time of observation. Both of the PHA lectins did not display remarkable scattered spot or single spot formation. Even clustering was only detected to a limited extend. Employing LCA, fluorescence accumulation in the form of tiny points around the cell boundaries could be monitored, specifically along long membrane stretches. This signal type is displayed in FIG. 9. Examining CHO/PAR-2 cells, all lectins that produced clear labeling pattern on fixed cells were found to proceed with signal transformation according to the scheme. After 60 minutes centered spots or at least discrete areas of fluorescence were detectable in the cells with all candidates. PHA-L and PHA-E generated a strong signal contrast between the relatively unstained nucleus area and its fluorescing perimeter. Cell surface fluorescence was completely clustered into small point signals. At a later stage, spot signals emerged, too, as depicted in FIG. 10. An overview of staining patters obtained with the three cell lines is given in Table 6. In all experiments, fluorescence intensity, and hence signal resolution, decreased over time. Some lectins, like PHA-L and PHA-E, delivered fairly stable signals. However, lectins that gave a high background in fixed cell experiments also showed a strong blurring of the signal pattern. Summarizing the results, it can be stated that fluorescence signals from live cell labeling differed from those obtained with fixed cells. Fluorescent lectins were clearly guided by cellular dynamics and exhibited several types of transformation over a time course of two hours and more. A very prominent signal pattern associated with all cell types was the accumulation of fluorescence to form an intense spot in the perinuclear region. As observed with fixed cells, the resolution of the fluorescent signal as well as signal behavior was found to be dependent on lectin candidate and cell type. Moreover, elevated lectin concentrations increased signal dynamics and seem to affect cell morphology.

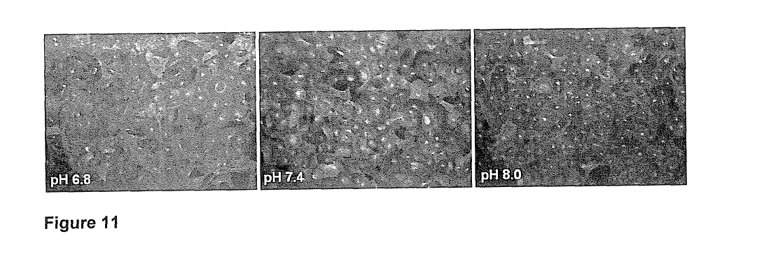

It is preferred for an assay that detects a signal translocation from the membrane into the cytoplasm, as expected for a receptor internalization assay, to have a low background signal while the cell is unstimulated. Evidently, the spot formation represented a background that is not preferred. Rather, it resembled the type of signal that was desired under stimulated conditions. In this regard, the origin of the spot signal was of crucial interest since identification of the cause could offer a way to circumvent it. In a preferred embodiment, a sWGA, as the most versatile candidate, was employed with U2OS cells to discover possible environmental factors that had an impact on signal development. This was done by varying the experimental conditions such as temperature level, pH level, and incubation periods. In these experiments, slight deviations from the reported time course could be recorded, but the general progression of spot formation was found to be constant. Yet, the most striking influence could be ascribed to the pH level since the reduction of the pH to 6.8 caused a 20 minute delay in spot formation, whereas an increase to 8.0 accelerated it. This context is depicted in FIG. 11. In this regard, the extracellular pH level was confirmed a factor that influenced the signal development. According to a preferred embodiment of the present invention, the pH is chosen in such a way as to minimize spot generation in unstimulated cells so as to reduce background signal.

### 1.3 Serum Supplements in Live Cell Labeling

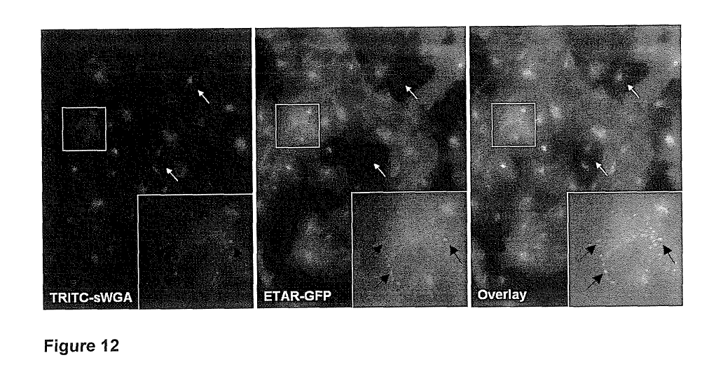

The protocol for live cell labeling included a period of serum deprivation in order to avoid interference from exogenous stimuli. Nonetheless, it could not ruled out that certain substances resisted the washing procedure and remained potentially active during the starvation. In this approach, a defined addition of serum supplement in the starvation medium was carried out to test whether it in some way affected the fluorescence signal. Therefore, Opti-MEM medium (11058; Gibco—Invitrogen, Karlsruhe, Germany) was used for starvation, which constitutes a reduced serum medium on DMEM basis for transfection purposes, containing only insulin and transferrin as protein components at a maximum level of 15 μg/ml. For these studies sWGA and U2OS/ETAR-GFP cells were used. Utilizing Opti-MEM, the signal was found to be much weaker and the characteristic fluorescence accumulation in the center was diminished in favor of numerous smaller spots scattered over the cell. Furthermore, when the ETAR-GFP signal that featured a homogeneous distribution over the cell surface under standard conditions was compared to the signal obtained after Opti-MEM incubation, the pattern closely resembled the one in the TRITC-sWGA channel. Large spots as well as small spots around the nucleus area seemed to be identical in both of the fluorescence channels and were, in fact, found to co-localize in a superimposition of the two images. This context is shown in FIG. 12. Hence, it was confirmed that in a preferred embodiment, the addition of insulin to the incubation medium positively influenced the development of a distinct lectin signal.

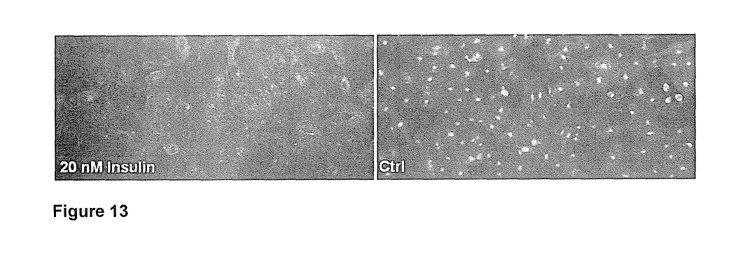

Insulin was a key protein component in the reduced serum medium. Hence, the impact of insulin was selected for further investigations. These showed that addition of 20 nM insulin to the assay medium in the labeling and chase incubations resulted in a complete suppression of spot formation. In fact, it reinforced the signal type described for reduced serum medium. Images displayed in FIG. 13 depict this effect. Therefore, it is preferred to add serum factors, specifically insulin, to the incubation medium to prevent or reduce spot formation.

These results underscored that internalization of fluorescent lectins by endocytosis was a very plausible scenario. They further implicated that intracellular trafficking was the underlying principle of signal transformation. In order to test this hypothesis, experiments were carried out, which focused on the initial step in endocytosis: The internalization of cargo from the plasma membrane.

### 1.4 Internalization Pathway Analysis in U2OS/ETAR-GFP Cells

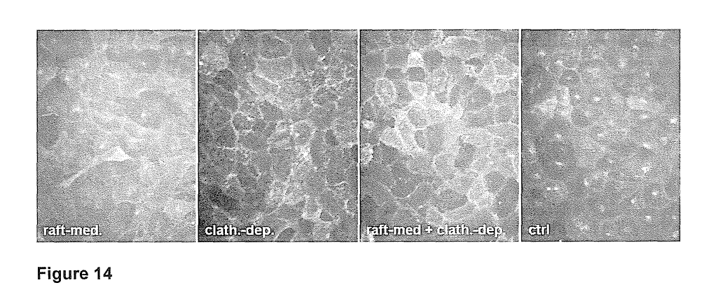

Based on the protocol established for live cell labeling, lectin internalization via raft-dependent, clathrin-mediated, or non-raft-non-clathrin-mediated endocytosis was examined by inhibition of the respective pathway. In this regard, CLAME can be inhibited by subjecting the cells to a brief hypotonic shock and subsequently utilizing a potassium depleted medium, whereas RDE can be abolished by extraction of cholesterol from the plasma membrane using β-methyl-cyclodextrin. Cholesterol is a major component of raft-domains and caveaolae. Therefore, extraction disrupts these domains and prevents internalization of associated proteins and lipids. A combination of both methods should result in a complete cessation of endocytosis via both pathways. In these experiments, it was found that inhibition of raft-dependent pathways did not impede the typical spot formation, albeit the signal was diminished in intensity. Additionally, an elevated level of residual, homogeneous fluorescence at the cell surface could be detected. In contrast, inhibition of the clathrin-mediated pathway displayed a clustered type of fluorescence at cell surface without spot formation within the time of observation. Moreover, combination of inhibitions resulted in a suppression of the spot signal, similar to clathrin-mediated inhibition. Furthermore, it featured a faintly clustered, but mainly homogeneous type of cell surface fluorescence. All signal types are depicted in FIG. 14.

To verify the intended inhibition, Alexa-Fluor® 488-transferrin, a standard marker of the clathrin-mediated pathway, was used as a control for the respective. Here, signals were detected in the perinuclear region of cells under all conditions but those involving inhibition of the clathrin-dependent pathway. Thus, inhibition of CLAME was regarded as successful. Yet, an aspect that limited further conclusions was that no control was available for the raft-dependent pathway. Generally SV40 virus gets employed since it is known to use this internalization route exclusively. Even though the effect of cholesterol extraction is well characterized, affection of the clathrin-mediated pathway can generally not be ruled out. The ETAR-GFP signal was not initially intended to be a read-out signal in this experiment, but when GFP fluorescence was investigated, signals showed an identical behavior to the lectin signal under conditions of clathrin-mediated inhibition, whereas it was not affected under raft-mediated inhibition only. Summarizing the facts, it was found that analysis of the endocytosis pathways by which sWGA could enter U2OS cells yielded very indicative signal types: All pathway inhibitions reduced the total amount of non-homogeneous signals present. Additionally, the inhibition of the clathrin-mediated pathway caused the prevention of spot formation. Also, ETAR-GFP signals were affected by the measures taken for inhibition of CLAME.

1.5 Lectin Labeling of Live Cells with Receptor Targeted Stimulation

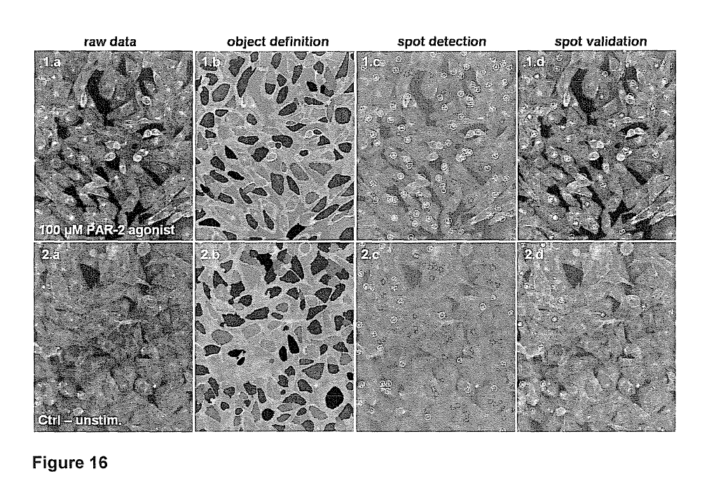

In order to test whether a specific internalization response induced by receptor stimulation could be inferred from the fluorescent signal, stimulation experiments were set up. Concerning the read-out, analysis of discrepancies in the chased lectin fluorescence signals was carried out comparing stimulated cells versus non-stimulated cells. In this regard, stimulation was defined as the addition of ligand precisely targeting the respective recombinant receptor in each of the three cell lines. With CHO/ETAR, no reference signal could be used as a parallel control. Hence, only unstimulated cells served as a reference. In these studies, only sWGA was tested due to the limited extend of the study and delivered weak spot signal upon stimulation through endothelin 1, which slightly preceded the signal of the unstimulated reference by four to eight minutes. However, signals could be discriminated within that time frame between minute 30 and 38 of the assay. These results seemed indicative. Stimulation induced different endocytosis kinetics. Conducting the same experiments with CHO/PAR-2 cells and sWGA, PHA-L, and GSL-I, they responded in a similar way as the ETAR cells. Upon stimulation with PAR-2 agonist peptide, early spot formation was observed for all lectins tested. sWGA signals could be discriminated the best, followed by PHA-L. In this assay, the time frame was found to lie between minute 30 and 40, including the 10 minute labeling interval prior to stimulation. In all assays that allowed for discrimination, a minor population of cells within the controls already exposed sequestered fluorescence or even discrete spots, but this effect could not erode a sound statistical significance of the stimulated signal. The result for PAR-2 was reproduced on several days and showed a random shifting tendency with respect to the emergence of spots in the control. Thus, the time frame was subject to a certain variation. FIG. 16 displays the result for sWGA on CHO/PAR-2 cells, which proved a good system with regard to the signal-to-noise ratio. This series of key experiments showed that with the two CHO cell lines a fluorescent response was measured, which could be ascribed to the stimulation of the recombinant receptor. It was observed with all lectins tested (CHO/ETAR with sWGA; CHO/PAR-2 with sWGA, PHA-L, and GSL-I). They all shared the same feature of early spot formation about 20 to 30 minutes after stimulation. In fact, these results show that the concept of the aimed at assay functions.

In order to improve discrimination between induced and background signal and thereby to allow for a better read-out, the assay protocol offered a suitable handle for modification. It is preferred for an application in receptor studies, especially in HCS, that the signal could also be read by automated microscopy and that significance of the induced signal could be assessed by software-based image processing.

1.6 Lectin Labeling of Live Cells with Modified Stimulation

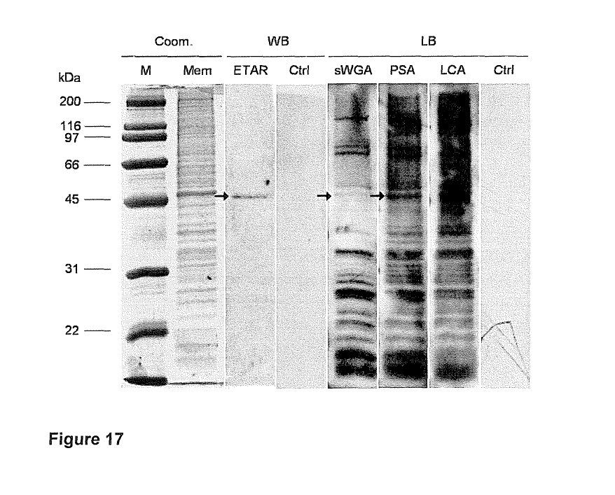

The receptor specific signal observed in preceding experiments offered only a small assay window to monitor the response. To improve signal discrimination, the protocol was modified and now featured stimulation at an earlier stage in the procedure, simultaneous with the lectin labeling. In the following, cell fixation served to arrest cells at desired points of signal development and permitted automated imaging on the Opera™ confocal fluorescence microscope without assay time restrictions. These investigations were conducted with sWGA on CHO/PAR-2 cells. The reported stimulation effect of the assay was shifted to an earlier point by including PAR-2 agonist already in the labeling medium. The time frame was thereby extended to almost 15 minutes, which clearly confirmed the effect. Confocal Opera microscopy of fixed cells captured a series of appropriate images for analysis with Acapella. A representative collection of images at subsequent stages is depicted in FIG. 17. It features a stimulation experiment and a control.

### 2. Lectin and Western Blotting for In Vitro Binding Analysis

In the following, the results of the lectin and western blotting for in vitro binding analysis are presented. An in vitro method was set up to verify recombinant receptor expression and to classify binding patterns of lectins to cell surface proteins. For the task given, a western blot-lectin blot combination was considered a powerful assay, most importantly, because comparison of the obtained probing results should be able to give insight into potential interactions between a lectin candidate and the receptor of interest.

### 2.1 Cell Membrane Preparation

In order to obtain a high quality sample to start with, a cell membrane preparation was conducted to obtain only cell membrane associated protein for the blotting experiments. Due to the results from the internalization assay obtained with the CHO cell lines and the limited capacity, only CHO/ETAR and CHO/PAR-2 cells were prepared. Cell yield after harvesting was 10.8*10⁷ cells and 2.15*10⁷ cells, respectively. In both preparations, a transparent pellet was visible after ultra-centrifugation, which was resuspended in 1 ml of buffer in the case of ETAR and in 0.5 ml in the case of PAR-2. Supernatant of the PAR-2 preparation was not discarded, but served as a control in succeeding experiments. The pellet obtained after ultracentrifugation was assumed to constitute cell membrane fragments. To verify a successful protein preparation, the protein content needed to be determined.

### 2.2 Determination of Protein Content

The protein concentration of the membrane preparations and the supernatant was measured employing a BCA assay. In addition to verifying the protein content, this information served to compare preparations and to normalize gel loads. In all assays run, BSA protein standards delivered a linear correlation between BSA concentration and absorbance (Abs) in the range between 0<Abs<1.5. Three values for the unknown samples were averaged to give a protein content of 2.04 mg/ml±0.09 mg/ml for the ETAR preparation and 2.15 mg/ml±0.05 mg/ml for the PAR-2 preparation. Moreover, the supernatant of the latter was determined to be 0.31 mg/ml±0.004 mg/ml. All samples contained considerable amount of protein. Furthermore, the supernatant concentration was found to be 14% of the membrane sample. To continue with the analysis, protein resolution by SDS PAGE was carried out.

### 2.3 SDS-Page of Membrane Samples

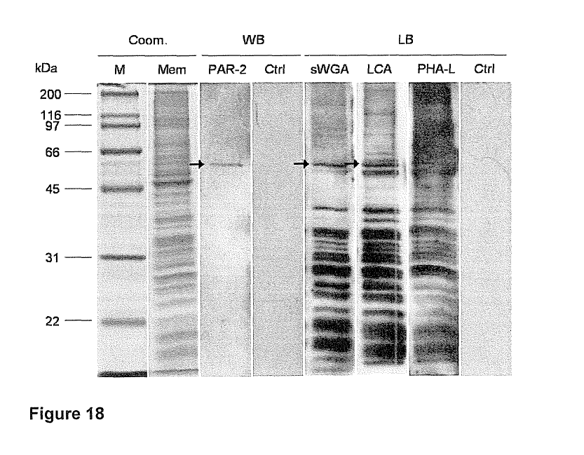

In order to resolve membrane samples for further staining and blotting procedures, an SDS-PAGE was conducted. Resolving 25 μg of membrane protein on 12% polyacrylamide gels by SDS-PAGE yielded a homogeneous pattern of bands as could be seen in the coomassie staining Bands were distributed over the whole length of the lanes. In both membrane samples, only one intensively stained band at 47 kDa stood out. In contrast, the supernatant of PAR-2 displayed a more heterogeneous blend of proteins with at least six striking bands, albeit bands in the spectrum below 40 kDa were hardly visible. When membrane sample and supernatant were normalized to 6.8 μg per lane, the supernatant fraction was found to be stained at least five times as intensively as the membrane fraction. Coomassie stainings of both of the membrane samples are shown in FIGS. 17 and 18. In this experiment, resolved membrane samples gave well visible lane patterns in the coomassie staining But in contrast, normalized samples of supernatant and membrane fraction featured a much higher staining intensity of the latter. This result seemed to question the reliability of the protein assay, however, it did not impede subsequent steps. In this regard, protein from the gels needed to be transferred onto a nitrocellulose membrane in order to detect the recombinant receptor and protein binding partners of lectins.

### 2.4 Blotting of Membrane Samples

The gels obtained by SDS-PAGE were transfered onto nitrocellulose membranes for the purpose of probing them later on. It was found that the Ponceau S staining of membranes displayed an identical band pattern as the coomassie staining and, thereby, implied a successful transfer. For succeeding experiments, only membrane slices were selected that exhibited an undisturbed dye pattern to avoid processing of transfer artifacts.

### 2.5 Probing of Membrane Samples

The final probing step was intended to give information about receptor expression and molecular weight using an antibody and about the spectrum of proteins that contained a target glycosylation using a lectin. Both procedures followed the same standardized western blotting protocol, albeit, for the lectin blots biotin-labeled lectins were used as primary detection reagents, which were in turn recognized by avidin-HRP as the secondary reagent. For the ETAR preparation an anti-ETAR-antibody was employed. It produced a clear, discrete band at 47 kDa after 10 minutes of exposure. This band was not present in the control without the primary antibody. A scan of the films is depicted in FIG. 18. For the lectin blots sWGA, PSA, and LCA were used. They all produced a quite similar pattern with at least 13 intensive bands within exposure times of one to three minutes. No signal was observed for the control with only avidin-HRP. Especially in the spectrum below 35 kDa, the marking pattern of the different lectins was almost identical. Above 35 kDa, bands were scarce and marking specificities differed a little from one another, especially with regard to the intensity. sWGA gave the best band contrast with hardly any background, followed by PSA, which displayed an increased background in the lane region above 50 kDa. The LCA signal was significantly corrupted by a background in the same spectrum that was even higher. A band at the same height as in the western could clearly be detected in the PSA lane. The sWGA lane also displayed a very faint band. Due to the high background, no statement can be made for LCA. Scans of the films are shown in FIG. 18. These were aligned according to the marker bands visible on the films owing to the ballpoint pen drawing. However, accuracy of alignment was limited by the given clearance of the marker bands. For the PAR-2 preparation an anti-PAR-2-antibody was utilized. A clear, separate band at 55 kDa was detected after 10 minutes of exposure. The control without primary antibody did not exhibit a band, neither was a band detected in the supernatant of the ultra-centrifugation. The probed membrane film and its control are depicted in FIG. 19. For lectin blotting, sWGA, LCA, and PHA-L were applied. The result was very similar to the one obtained with the ETAR sample. Distinct band patterns could be generated with the probes after one to five minutes of exposure, which were almost identical in the spectrum below 35 kDa. However, the pattern of PHA-L differed remarkably in three bands. Above 35 kDa, two clear bands were detected by sWGA, one at the same height as the western band. LCA displayed four intensive bands, one of them matching the height of the western band. The PHA-L signal in that spectrum suffered from a high background that prevented recognition of bands. Moreover, the control without biotinylated lectin did not give a signal. In the supernatant one very faint band at 70 kDa was present. Again, the highest contrast and the clearest bands were obtained with the sWGA probe followed by the LCA probe. The illustrated FIG. 19 depicts scans of the films. Scans of the supernatant are not shown. Furthermore, alignment was subject to the same limitation as in the ETAR case. Additionally, interesting information resulted from a comparison of the two preparations.

Aligning band patterns of identical detection methods from the two cell lines, it was striking that the coomassie lane patterns resembled one another. They featured identical bands between 31 and 45 kDa, a prominent one at 47 kDa and a weaker one at 100 kDa. Focusing on the sWGA blot, the two patterns showed significant deviations from each other. In the ETAR sample, only five bands stood out below 35 kDa, although the PAR-2 sample yielded at least nine striking bands. Furthermore, the remainder in the upper spectrum did not give a match at all. Moreover, differential labeling was also found for LCA. Summarizing the results, it can be stated that these detections delivered a fair amount of data and interesting outcomes, especially when putting individual probing experiments into context. The western blotting suggested that each of the antibodies was able to detect the respective antigen in the sample. Furthermore, lectins produced quite similar band patterns within the same sample, albeit differential labeling was also found. Furthermore, lectin lanes between CHO membrane samples differed significantly, even though coomassie staining exhibited an analogous pattern for total protein in both samples. Thus, individual lectin patterns were more similar to each other within one membrane preparation than compared to the corresponding pattern in the other preparation. Moreover, some lectins also displayed a band at the same molecular weight as the antibody used for that sample. In this regard, sWGA should be emphasized here as the critical link to the internalization assay. Only a very faint band could be detected at the height of the respective western band in the ETAR sample, whereas an intense band was observed with the PAR-2 sample.

### 3. Summary of Results

In summary, the above experiments on lectins-cell interactions provided new insights and options for applications in the field of endocytosis assays. It proved fluorescent lectin candidates to be valuable labeling agents for cell membranes, exhibiting high affinity and specificity. The signal patterns and transformations obtained in experiments with fixed cells and live cells, respectively, were ascribed to lectins-glycoprotein and lectin-glycosphingo lipid interactions. These complexes were subsequently internalized via several endocytosis pathways simultaneously, albeit to different extents, depending on the lectin and the cell type employed. It could be concluded that very efficient, constitutive CLAME was responsible for the emergence of lectin-substrate complexes in a perinuclear compartment, presumably the TNG or the recycling endosome. Therefore, it was deduced that receptor stimulation had a crucial impact on trafficking since insulin addition led to a significant change in the endocytic signal response. Moreover, co-localization of signals indicated that lectin and GPCR internalization routes at least partially coincided. GPCR-specific endocytic responses were obtained with two cell lines, where fluorescence accumulated in the perinuclear compartment within the expected interval for GPCR endocytosis. Automated fluorescence microscopy and image processing successfully verified significance of the signal and proved it an appropriate read-out parameter for quantification that is compatible with assay scale-up.

In the following, a description of the figures is provided.

FIG. 1: Overview of the major endocytosis routes including the compartment and vesicle markers, respectively, highlighted in dark blue. Key compartments are the sorting endosome as a port for vesicles from clathrin-mediated and non-clathrin-non-caveolae endocytosis, as well as from macropinocytosis and the golgi as the interface with the de novo synthesis route. The figure was taken from Sieczkarski and Whittaker (2002).

FIG. 2: Assembly for semi-dry blotting; gel and nitrocellulose membrane are embraced by 3 mm Whatman papers soaked in diverging blotting buffers: Whatman paper in Anode Buffer I (AB I) and Anode Buffer II (AB II), AB II soaked nitrocellulose membrane, gel, Whatman paper in Cathode Buffer (CB).

FIG. 3: Fixed U2OS/ETAR-GFP cells labeled with LCA; 40×

FIG. 4: Fixed CHO/ETAR cells labeled with PHA-L; 20×

FIG. 5: Fixed CHO/PAR-2 cells labeled with GSL-I; 20×

FIG. 6: Irritating effect of SJA to U2OS/ETAR-GFP cells at concentrations of 5 nM, 10 nM, and 20 nM versus the control (Ctrl); phase contrast; 10×

FIG. 7: Overlay of sWGA signals (red) and Hoechst signals for nucleus staining (blue) with U2OS cells; arrows point out concave inversions of nuclei directed towards spot-like lectin signals; 20×

FIG. 8: Live U2OS/ETAR-GFP cells labeled with WGA; 42 min, 20×

FIG. 9: Live CHO/ETAR live cells labeled with LCA; 62 min, 20×

FIG. 10: Live CHO/PAR-2 cells labeled with PHA-L; 76 min, 20×

FIG. 11: Live U2OS/ETAR-GFP cells labeled with sWGA at different pH levels. A delay of 20 minutes in spot formation was observed at pH 6.8 compared to pH 8.0; Images taken at 42 min; 20×

FIG. 12: Labeled U2OS/ETAR-GFP cells displaying the sWGA signal (red) and the ETAR-GFP signal (green), which were superimposed (red and green). Large squares represent a 3-fold magnification of small squares to visualize co-localization of spots indicated by black arrows. White arrows point out cells without GFP signal as controls for signal co-localization due to channel-crosstalk; 40×

FIG. 13: sWGA labeled U2OS/ETAR-GFP cells with 20 nM insulin supplemented to the assay medium for labeling and chase incubations and the control (Ctrl); 60 min, 20×

FIG. 14: sWGA labeled U2OS cells under inhibition of major endocytosis pathways: raft-mediated (raft-med.), clathrin-dependent (clath.-dep.), and a combination of both (raft-med.+clath.-dep.) versus the control (Ctrl); 35 min, 20×

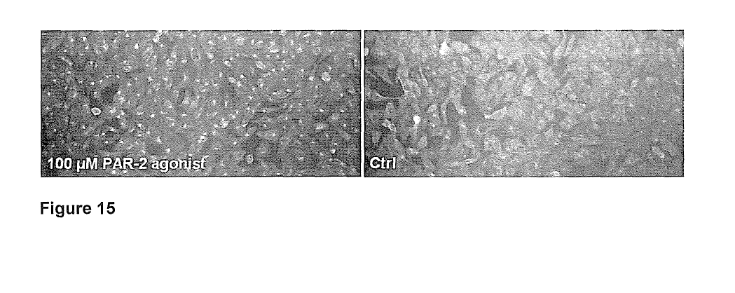

FIG. 15: sWGA labeled CHO/PAR-2 cells with receptor targeted stimulation of 100 μl PAR-2 agonist peptide versus the control (Ctrl); 42 min, 20×

FIG. 16: Representative collection of images at subsequent stages of the Acapella spot detection. processing stimulated cells (Series 1) versus the unstimulated control (Series 2): a. image raw data; b. object definition based on nucleus and cytoplasm detection (random color distribution); c. spot detection (white: validated, green: discarded by contrast criterion, red: discarded by contrast and spot-to-cell intensity criterion); d. validated spots (random color distribution); 20×

FIG. 17: Scans of different detection methods after ETAR membrane sample resolution by SDS-PAGE; Coomassie staining (Coom.) with marker (M) and membrane sample (Mem); Western blot (WB) with anti-ETAR-antibody (ETAR) and control without 1° antibody (Ctrl); Lectin blot (LB) with sWGA, PSA, LCA, and the control with avidin-HRP only (Ctr1); arrows indicate bands at 47 kDa.

FIG. 18: Scans of different detection methods after PAR-2 membrane sample resolution by SDS-PAGE; Coomassie staining (Coom.) with marker (M) and membrane sample (Mem); Western blot (WB) with anti-PAR-2-antibody (PAR-2) and control without 1° antibody (Ctrl); Lectin blot (LB) with sWGA, LCA, PHA-L, and the control with avidin-HRP only (Ctrl); arrows indicate bands at 55 kDa.

In the following, the tables mentioned above are provided:

TABLE 1
Media formulations essential for the employed cell lines

| Cell Line | Basal Medium | Serum | Antibiotics |
|---|---|---|---|
| U2OS/ | DMEM:F12 (1:1) with | 10% FCS | 500 μg/ml G418 salt |
| ETAR-GFP | L-glutamine | (Sigma | solution |
| | (Gibco 331331-028). | F9665) | (Sigma 8168) |
| CHO-K1/ | DMEM:F12 (1:1) with | 10% FCS | 500 μg/ml G418 salt |
| ETAR | L-glutamine | (Sigma | solution |
| | (Gibco 331331-028). | F9665) | (Sigma 8168) |
| CHO-K1/ | F12 nutrient mixture | 10% FCS | 100 units penicillin + |
| PAR-2 | (Sigma N6658) | (Sigma | 100 μg/ml |
| | | F9665) | streptomycin. |
| | | | (Sigma P4458), |
| | | | 400 μg/ml hygromycin |
| | | | (Sigma 0654) |

TABLE 2
List of employed lectins with species derived name, abbreviation, recognized native
glyco-epitope, and inhibitory monosaccharide. All information provided by Vector Labs
(Vector Labs Homepage); asterisk (*) marked entries from Spicer and Schulte (1992).

| | | Recognized Glyco- | |
|---|---|---|---|
| Lectin | Abbr. | Epitope | Inhibitory Monosaccharide |
| Griffonia | GSL I | α-N-acetylgalactosamine | 200 mM galactose + 200 mM |
| (Bandeiraea) | | and α-galactose | N-acetylgalactosamine |
| simplicifolia lectin | | | |
| Pisum sativum | PSA | α-linked mannose-glycans | 200 mM α-methyl |
| (garden pea) agglutin | | containing N-acetyl- | mannoside + 200 mM α- |
| | | chitobiose-linked α- | methyl glucoside |
| | | fucose | |
| Lens culinaris (lentil) | LCA | α-linked mannose | 200 mM α-methyl |
| agglutin | | | mannoside + 200 mM α- |
| | | | methyl glucoside |
| Phaseolus vulgaris | PHA-L | triantennary complex | 100 mM acetic acid |
| (red kidney bean) | | oligosaccharides with N- | |
| leucoagglutin | | acetyllucosamine β-1,2 | |
| | | mannose residues* | |
| Phaseolus vulgaris | PHA-E | bisected complex | 100 mM acetic acid |
| (red kidney bean) | | oligosaccharides* | |
| erythroagglutin | | | |
| Sophora japonica | SJA | terminal N-acetyl- | 200 mM N-acetylgalactosamine |
| (Japanese pagoda | | galactosamine and | |
| tree) agglutin | | galactose residues, | |
| | | preferentially binding to | |
| | | β-anomers | |
| Triticum vulgaris | sWGA | N-acetylglucosamine | 500 mM N- |
| (wheat germ) | | | acetylglucosamine with salt |
| agglutin, succinylated | | | |

TABLE 3
Parameters for Opera based fluorescence image capture consisting of
wavelength of excitation laser (λ_{Ex}), captured emission
wavelength spectrum (Δλ_{Trans}), exposure time
(t_{Exp}) and binning mode.

| Fluorophore | λ_{Ex} [nm] | Δλ_{Trans} [nm] | T_{Exp} [ms] | Binning Mode [—] |
|---|---|---|---|---|
| TRITC | 532 | 585/40 | 4000 | 2 × 2 |
| DRAQ5 ™ | 635 | 690/50 | 800 | 2 × 2 |

TABLE 4
Staining patterns of the lectins on fixed cells of
the three employed cell lines

| | Staining of | Staining of | Staining of |
|---|---|---|---|
| Lectin | U2OS/ETAR-GFP | CHO/ETAR | CHO/PAR-2 |
| sWGA | cell surface | cell surface | cell surface |
| PHA-L | cell surface | cell surface | cell surface |
| PHA-E | cell surface | cell surface | cell surface |
| LCA | cell surface | cell surface | weak cell surface |
| PSA | cell surface | | weak cell surface |
| GSL-I | weak single cell | | weak cell surface |
| SJA | weak spots on cell | | |

TABLE 5
Scheme of signal transformation on live cells after
labeling with fluorescent lectins.

| | Time | |
|---|---|---|
| | Course | |
| Phase | [min] | Characteristic Fluorescence Signal |
| 1 | 10-20 | Sequestering of signals to small points over the whole |
| | | cell surface, pecifically at cell boundaries. |
| 2 | 20-30 | Formation of lager spots from small points at more |
| | | centered positions n the cell with a concomitant |
| | | decrease in cell surface fluorescence. |
| 3 | 30-60 | Unification of spots at one central position per the cell. |
| 4 | 30 | Remaining of homogeneous fluorescence on the whole |
| | | cell surface at varying degrees; persistence of clustered, |
| | | intensive fluorescence at the outer cell boundaries |
| | | and cell-cell junctions. |
| 5 | 150 | Partial distribution into small spots around a fluorescent |
| | | spot signal; only observable over longer periods. |

TABLE 6
Live cell stainings obtained with the lectins
on the three employed cell lines

| | | | Live cell |
|---|---|---|---|
| | Live cell staining of | Live cell staining of | staining of |
| Lectin | U2OS/ETAR-GFP | CHO/ETAR | CHO/PAR-2 |
| sWGA | spot formation | spot formation | spot formation |
| PHA-L | spot formation | cell surface, sequestered | spot formation |
| PHA-E | spot formation | cell surface, sequestered | spot formation |
| LCA | spot formation | cell boundary, points | spot formation |
| PSA | spot formation | | |
| GSL-I | small, scattered | | |
| | spots | | |
| SJA | scattered spots | | |

References to the following articles have been made above:
Bhowmick, N., Narayan, P., and Puett, D. (1998) The endothelin subtype A receptor undergoes agonist- and antagonist-mediated internalization in the absence of signaling. Endocrinology 139: 3185-3192.
Bohm, S. K., Khitin, L. M., Grady, E. F., Aponte, G., Payan, D. G., and Bunnett, N. W. (1996) Mechanisms of desensitization and resensitization of proteinase-activated receptor-2. J. Biol. Chem. 271: 22003-22016.
Bousso-Mittler, D., Galron, R., and Sokolovsky, M. (1991) Endothelin/sarafotoxin receptor heterogeneity: evidence for different glycosylation in receptors from different tissues. Biochem. Biophys. Res. Commun. 178: 921-926.
Brodsky, F. M., Chen, C. Y., Knuehl, C., Towler, M. C., and Wakeham, D. E. (2001) Biological basket weaving: formation and function of clathrin-coated vesicles. Annu. Rev. Cell Dev. Biol. 17: 517-568.
Ceresa, B. P., Kao, A. W., Santeler, S. R., and Pessin, J. E. (1998) Inhibition of clathrin-mediated endocytosis selectively attenuates specific insulin receptor signal transduction pathways. Mol. Cell Biol. 18: 3862-3870.
Chun, M., Liyanage, U. K., Lisanti, M. P., and Lodish, H. F. (1994) Signal transduction of a G protein-coupled receptor in caveolae: colocalization of endothelin and its receptor with caveolin. Proc. Natl. Acad. Sci. U.S. A 91: 11728-11732.
Chun, M., Lin, H. Y., Henis, Y. I., and Lodish, H. F. (1995) Endothelin-induced endocytosis of cell surface ETA receptors. Endothelin remains intact and bound to the ETA receptor. J. Biol. Chem. 270: 10855-10860.
Comley, J. (2005) High content screening. Drug Discovery World Summer 2005: 31-53.
Compton, S. J., Sandhu, S., Wijesuriya, S. J., and Hollenberg, M. D. (2002) Glycosylation of human proteinase-activated receptor-2 (hPAR2): role in cell surface expression and signalling. Biochem. J. 368: 495-505.
Danguy, A., Decaestecker, C., Genten, F., Salmon, I., and Kiss, R. (1998) Applications of lectins and neoglycoconjugates in histology and pathology. Acta Anat. (Basel) 161: 206-218.
Dove, A. (2003) Screening for content—the evolution of high throughput. Nat. Biotechnol. 21: 859-864.
Drews, J. (2000) Drug discovery: a historical perspective. Science 287: 1960-1964.
Dube, D. H. and Bertozzi, C. R. (2005) Glycans in cancer and inflammation—potential for therapeutics and diagnostics. Nat. Rev. Drug Discov. 4: 477-488.
Eglen, R. M. (2005) An overview of high throughput screening at G protein coupled receptors. Frontiers in Drug Design & Discovery 1:97-111.
Futerman, A. H. and Hannun, Y. A. (2004) The complex life of simple sphingolipids. EMBO Rep. 5: 777-782.
Gabius, H. J. (1997) Animal lectins. Eur. J. Biochem. 243: 543-576.
Gaidarov, I., Santini, F., Warren, R. A., and Keen, J. H. (1999) Spatial control of coated-pit dynamics in living cells. Nat. Cell Biol. 1: 1-7.
Geyer, H. and Geyer, R. (1998) Strategies for glycoconjugate analysis. Acta Anat. (Basel) 161: 18-35.
Griffiths, G., Back, R., and Marsh, M. (1989) A quantitative analysis of the endocytic pathway in baby hamster kidney cells. J. Cell Biol. 109: 2703-2720.
Heinrich, E. L., Welty, L. A., Banner, L. R., and Oppenheimer, S. B. (2005) Direct targeting of cancer cells: A multiparameter approach. Acta Histochem. 107: 335-344.
Holman, G. D. and Sandoval, I. V. (2001) Moving the insulin-regulated glucose transporter GLUT4 into and out of storage. Trends Cell Biol. 11: 173-179.
Innamorati, G., Le Gouill, C., Balamotis, M., and Birnbaumer, M. (2001) The long and the short cycle. Alternative intracellular routes for trafficking of G-protein-coupled receptors. J. Biol. Chem. 276: 13096-13103.
Johannes, L. and Lamaze, C. (2002) Clathrin-dependent or not: is it still the question? Traffic. 3: 443-451.
Koo, B. H., Chung, K. H., Hwang, K. C., and Kim, D. S. (2002) Factor Xa induces mitogenesis of coronary artery smooth muscle cell via activation of PAR-2. FEBS Lett. 523: 85-89.
Lanctot, P. M., Leclerc, P. C., Clement, M., Auger-Messier, M., Escher, E., Leduc, R., and Guillemette, G. (2005) Importance of N-glycosylation positioning for cell-surface expression, targeting, affinity and quality control of the human AT1 receptor. Biochem. J. 390: 367-376.
Le Roy, C. and Wrana, J. L. (2005) Clathrin- and non-clathrin-mediated endocytic regulation of cell signalling. Nat. Rev. Mol. Cell Biol. 6: 112-126.
Leconte, I., Carpentier, J. L., and Clauser, E. (1994) The functions of the human insulin receptor are affected in different ways by mutation of each of the four N-glycosylation sites in the beta subunit. J. Biol. Chem. 269: 18062-18071.
Lodish, H. F., Berk, A., Zipursky, S. L., Matsudaira, P., Baltimore, D., and Darnell, J. (2000) Molecular cell biology. 4. Edition. W.H. Freeman. New York, U.S.A.
Lottspeich, F. and Zorbas, H. H. (1998) Bioanalytik. 1. Edition. Spektrum Verlag. Berlin-Heidelberg, Germany
McDonald, P. H., Chow, C. W., Miller, W. E., Laporte, S. A., Field, M. E., Lin, F. T. et al. (2000) Beta-arrestin 2: a receptor-regulated MAPK scaffold for the activation of JNK3. Science 290: 1574-1577.
Milligan, G. (2002) Strategies to identify ligands for orphan G-protein-coupled receptors. Biochem. Soc. Trans. 30: 789-793.
Mitchison, T. J. (2005) Small-molecule screening and profiling by using automated microscopy. Chembiochem. 6: 33-39.
Mukherjee, S., Ghosh, R. N., and Maxfield, F. R. (1997) Endocytosis. Physiol Rev. 77: 759-803.
Mundell, S. J., Loudon, R. P., and Benovic, J. L. (1999) Characterization of G protein-coupled receptor regulation in antisense mRNA-expressing cells with reduced arrestin levels. Biochemistry 38: 8723-8732.
Murrell, M. P., Yarema, K. J., and Levchenko, A. (2004) The systems biology of glycosylation. Chembiochem. 5: 1334-1347.
Ostrom, R. S. and Insel, P. A. (2004) The evolving role of lipid rafts and caveolae in G protein-coupled receptor signaling: implications for molecular pharmacology. Br. J. Pharmacol. 143: 235-245.
Paasche, J. D., Attramadal, T., Kristiansen, K., Oksvold, M. P., Johansen, H. K., Huitfeldt, H. S. et al. (2005) Subtype-specific sorting of the ETA endothelin receptor by a novel endocytic recycling signal for G protein-coupled receptors. Mol. Pharmacol. 67: 1581-1590.
Pelkmans, L. and Helenius, A. (2002) Endocytosis via caveolae. Traffic. 3: 311-320.
Reitman, M. L., Trowbridge, I. S., and Kornfeld, S. (1980) Mouse lymphoma cell lines resistant to pea lectin are defective in fucose metabolism. J. Biol. Chem. 255: 9900-9906.
Roosterman, D., Schmidlin, F., and Bunnett, N. W. (2003) Rab5a and rab11a mediate agonist-induced trafficking of protease-activated receptor 2. Am. J. Physiol Cell Physiol 284: C1319-C1329.
Roth, M. G., Doyle, C., Sambrook, J., and Gething, M. J. (1986) Heterologous transmembrane and cytoplasmic domains direct functional chimeric influenza virus hemagglutinins into the endocytic pathway. J. Cell Biol. 102: 1271-1283.
Sandvig, K., Olsnes, S., Brown, J. E., Petersen, O. W., and van Deurs, B. (1989) Endocytosis from coated pits of Shiga toxin: a glycolipid-binding protein from Shigella dysenteriae 1. J. Cell Biol. 108: 1331-1343.
Sandvig, K. and van Deurs, B. (2002) Transport of protein toxins into cells: pathways used by ricin, cholera toxin and Shiga toxin. FEBS Lett. 529: 49-53.
Santini, F. and Keen, J. H. (1996) Endocytosis of activated receptors and clathrin-coated pit formation: deciphering the chicken or egg relationship. J. Cell Biol. 132: 1025-1036.
Sharon, N. and Lis, H. (2004) History of lectins: from hemagglutinins to biological recognition molecules. Glycobiology 14: 53R-62R.
Shenoy, S. K. and Lefkowitz, R. J. (2003) Multifaceted roles of beta-arrestins in the regulation of seven-membrane-spanning receptor trafficking and signalling. Biochem. J. 375: 503-515.
Shoemaker, R. H., Scudiero, D. A., Melillo, G., Currens, M. J., Monks, A. P., Rabow, A. A. et al. (2002) Application of high-throughput, molecular-targeted screening to anticancer drug discovery. Curr. Top. Med. Chem. 2: 229-246.
Sieczkarski, S. B. and Whittaker, G. R. (2002) Dissecting virus entry via endocytosis. J. Gen. Virol. 83: 1535-1545.
Simons, K. and Ikonen, E. (1997) Functional rafts in cell membranes. Nature 387: 569-572.
Simonsen, A., Wurmser, A. E., Emr, S. D., and Stenmark, H. (2001) The role of phosphoinositides in membrane transport. Curr. Opin. Cell Biol. 13: 485-492.
Sonnichsen, B., De Renzis, S., Nielsen, E., Rietdorf, J., and Zerial, M. (2000) Distinct membrane domains on endosomes in the recycling pathway visualized by multicolor imaging of Rab4, Rab5, and Rab11. J. Cell Biol. 149: 901-914.
Spicer, S. S. and Schulte, B. A. (1992) Diversity of cell glycoconjugates shown histochemically: a perspective. J. Histochem. Cytochem. 40: 1-38.
Tarasova, N. I., Stauber, R. H., Choi, J. K., Hudson, E. A., Czerwinski, G., Miller, J. L. et al. (1997) Visualization of G protein-coupled receptor trafficking with the aid of the green fluorescent protein. Endocytosis and recycling of cholecystokinin receptor type A. J. Biol. Chem. 272: 14817-14824.
Thomsen, P., Roepstorff, K., Stahlhut, M., and van Deurs, B. (2002) Caveolae are highly immobile plasma membrane microdomains, which are not involved in constitutive endocytic trafficking Mol. Biol. Cell 13: 238-250.
Tsai, B., Gilbert, J. M., Stehle, T., Lencer, W., Benjamin, T. L., and Rapoport, T. A. (2003) Gangliosides are receptors for murine polyoma virus and SV40. EMBO J. 22: 4346-4355.
Tuan, T. L. and Grinnell, F. (1988) Wheat-germ-agglutinin and Ricinus communis-agglutinin-binding sites of BHK cells compared with each other and with 140 kDa fibronectin receptors. Biochem. J. 251: 269-277.
Vector Labs Homepage—Lectins (2005)
http://www.vectorlabs.com/products. asp?catID=58&locID=146
Vector Laboratories, Burlingame, Calif., U.S.A. last accessed: Nov. 28, 2005
Wilde, A., Beattie, E. C., Lem, L., Riethof, D. A., Liu, S. H., Mobley, W. C. et al. (1999) EGF receptor signaling stimulates SRC kinase phosphorylation of clathrin, influencing clathrin redistribution and EGF uptake. Cell 96: 677-687.

1. A method for monitoring a cell surface molecule and its potential internalisation into a cell, on the surface of which is located said cell surface molecule of interest, comprising:
(a) providing a sample carrier containing one or a plurality of cells which cell(s) possess a cell surface molecule of interest,
(b) adding a detectable lectin or lectin derivative to the cell(s), which binds to the cell surface molecule of interest, and
(c) monitoring the cell surface molecule of interest and its potential internalisation by detecting the lectin or lectin derivative.

2. A method for monitoring the internalisation of a cell surface molecule of interest into a cell, on the surface of which is located said cell surface molecule of interest, comprising:
(a) providing a sample carrier containing one or a plurality of cells which cell(s) possess a cell surface molecule of interest,
(b) adding a detectable lectin or lectin derivative to the cell(s), which binds to diverse cell surface molecules including the cell surface molecule of interest,
(c) stimulating the internalisation of the cell surface molecule of interest, and
(d) monitoring the internalisation of the cell surface molecule of interest by detecting the lectin or lectin derivative.

3. The method according to claim 1 wherein the internalisation of the cell surface molecule of interest is stimulated by adding a chemical compound or ligand to the cellular sample.

4. The method according to claim 1 wherein the degree of internalisation is determined by comparing the amount of detectable lectin or lectin derivative bound to the cell surface before and after stimulation of internalisation.

5. The method according to claim 1 wherein the degree of internalisation is determined by comparing the amount of detectable lectin or lectin derivative inside the cell before and after stimulation of internalisation.

6. The method according to claim 1 wherein the cell surface molecule of interest comprises a protein or a lipid molecule.

7. The method according to claim 6 wherein the protein or lipid molecule comprises a lectin or lectin derivative binding site.

8. The method according to claim 6 wherein the protein molecule is a cell surface receptor.

9. The method according to claim 8 wherein the cell surface receptor is a G-protein coupled receptor, a receptor tyrosine kinase, an ion channel, a cell adhesion molecule, a hormone receptor, a cytokine receptor, a chemokine receptor, a growth factor receptor, a neurotransmitter receptor, a lipoprotein receptor, a vitamin receptor, a viral binding receptor, a bacterial-interacting receptor, an antibody receptor, or a complement-binding receptor.

10. The method according to claim 6 wherein the lipid molecule is a glycolipid, a glycoglycerolipid, a glycoshingolipid, a glycophosphatidylinositol, a psychosine, a glycoglycerolipid, a ceramide, a monoglycosylceramide, a diosylceramide, a ganglioside, a glycuronosphingolipid, a sulfoglycoshingolipid, or a phosphonoglycosphingolipid.

11. The method according to claim 1 wherein the detectable lectin or lectin derivative is luminescently, fluorescently, or radioactively labelled.

12. The method according to claim 1 wherein the cell surface molecule of interest is a protein which is over-expressed in the cell.

13. The method according to claim 1 wherein the cell comprising the cell surface molecule of interest is a wild-type cell.

14. The method according to claim 1 wherein non-receptor mediated fluid-phase endocytosis processes are compressed by applying a medium comprising a background reducing agent.

15. The method according to claim 1 wherein the detectable lectin or lectin derivative is monitored by microscopy.

16. The method according to claim 15 wherein the microscopy is confocal microscopy.

17. The method according to claim 11 wherein the detection of the internalisation of the cell surface molecule to which a luminescently or fluorescently labeled lectin or lectin derivative is bound is performed by measuring a decrease of luminescence, preferably or fluorescence on the cell surface membrane.

18. The method according to claim 11 wherein the detection of the internalisation of the cell surface molecule to which a luminescently or fluorescently labeled lectin or lectin derivative is bound is performed by measuring an increase of luminescence or fluorescence within the cell.

19. The method according to claim 11 wherein the detection of the internalisation of the cell surface molecule to which a radioactively labelled lectin or lectin derivative is bound is performed by measuring a decrease of radioactivity on the cell surface membrane and/or an increase of radioactivity within the cell.

20. The method according to claim 11 wherein the area of cytoplasmic compartments, the fluorescence intensity within cytoplasmatic compartments, and/or the number of cytoplasmic compartments comprising fluorescently labelled lectin or lectin derivative is determined.

21. The method according to claim 1 for identifying compounds that induce or inhibit the internalisation of cell surface molecules.

22. The method according to claim 1 for use in drug discovery and drug development.

23. (canceled)
